SEQUENCE LISTING (6 sequences)

SEQ ID NO: 1 - DNA, 1921 bp, Homo sapiens
  misc_feature 1307, 1332: n = A,T,C or G
cttccaaagagcgactcttactgtttctcatggtgagaagacaatatttgctttctctttttcctttcttccggatgagaggctaagccataatagaaagaatggagaattattgattgaccgtctttattctgtgggctctgattctccaatgggaataccaagggatggttttccatactggaacccaaaggtaaagacactcaaagacagacatttttggcagagcatagatgaaaatggcaagttccctggctttccttctgctcaactttcatgtctccctcctcttggtccagctgctcactccttgctcagctcagttttctgtgcttggaccctctgggcccatcctggccatggtgggtgaagacgctgatctgccctgtcacctgttcccgaccatgagtgcagagaccatggagctgaagtgggtaagttccagcctaaggcaggtggtgaatgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctaatcttcacgtcgaagtgaagggttatgaggatggagggatccacctggagtgcaggtccaccggctggtacccccaaccccaaatacagtggagcaacgccaagggagagaacatcccagctgtggaagcacctgtggttgcagacggagtgggcctatatgaagtagcagcatctgtgatcatgaaaggcggctccggggagggtgtatcctgcatcatcagaaattccctcctcggcctggaaaagacagccagcatttccatcgcagaccccttcttcaggagcgcccagccctggatcgcagccctggcagggaccctgcctatcttgctgctgcttctcgccggagccagttacttcttgtggagactacagaaggaaataactgctctgtccagtgagatagaaagtgagcaagagatgaaagaaatgggatatgctgcaacagagcgggaaataagcctaagagagagcctccaggaggaactcaagaggaaaaaaatccagtacttgactcgtggagaggagtcttcgtccgataccaataagtcagcctgatgctctaatggaaaaatggccctcttcaagcctgcctgatttttcctgcatgggaagagcgcacatgtngccctgaggttcccttcccaggacngctccaggatcgagatcactgtgagtggttgtggagttaagacccctatggactccttcccagctgattatcagagccttagacccagcactccttggattggctctgcagagtgtcttggttgagagaataacgttgcagttcccacagggcatgtgactttgaaagagactaaaggccacactctgttaataatggggcacatatgtgttcccaccccacaaatgtgataagtgatcgtgcagccagagccagccttccttcagtcaaggtttccaggcagagcaaataccctagagattctctgtaatattggtaatttggatgaaggaagctagaagaattacagggatgtttttaatcccactatggactcagtctcctggaaaaggatctgtccactcctggtcattggtggatgttaaacccatattcctttcaactgctgcctgctagggaaaactgctcctcattatcatcactattattgctcaccactgtatcccctctactgggcaagtgcttgtcaagttctagttgttcaataaatttgttaataatgctgaaaaaaaaaaaaaaaaaaaaaaaa

SEQ ID NO: 2 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMKGGSGEGVSCIIRNSLLGLEKTASISIADPFFRSAQPWIAALAGTLPILLLLLAGASYFLWRLQKEITALSSEIESEQEMKEMGYAATEREISLRESLQEELKRKKIQYLTRGEESSSDTNKSA

SEQ ID NO: 3 - DNA, 2496 bp, Homo sapiens
gcgccctttcggtcaacatcgtagtccaccccctccccatccccagcccccggggattcaggctcgccagcgcccagccagggagccggccgggaagcgcgatgggggccccagccgcctcgctcctgctcctgctcctgctgttcgcctgctgctgggcgcccggcggggccaacctctcccaggacgacagccagccctggacatctgatgaaacagtggtggctggtggcaccgtggtgctcaagtgccaagtgaaagatcacgaggactcatccctgcaatggtctaaccctgctcagcagactctctactttggggagaagagagcccttcgagataatcgaattcagctggttacctctacgccccacgagctcagcatcagcatcagcaatgtggccctggcagacgagggcgagtacacctgctcaatcttcactatgcctgtgcgaactgccaagtccctcgtcactgtgctaggaattccacagaagcccatcatcactggttataaatcttcattacgggaaaaagacacagccaccctaaactgtcagtcttctgggagcaagcctgcagcccggctcacctggagaaagggtgaccaagaactccacggagaaccaacccgcatacaggaagatcccaatggtaaaaccttcactgtcagcagctcggtgacattccaggttacccgggaggatgatggggcgagcatcgtgtgctctgtgaaccatgaatctctaaagggagctgacagatccacctctcaacgcattgaagttttatacacaccaactgcgatgattaggccagaccctccccatcctcgtgagggccagaagctgttgctacactgtgagggtcgcggcaatccagtcccccagcagtacctatgggagaaggagggcagtgtgccacccctgaagatgacccaggagagtgccctgatcttccctttcctcaacaagagtgacagtggcacctacggctgcacagccaccagcaacatgggcagctacaaggcctactacaccctcaatgttaatgaccccagtccggtgccctcctcctccagcacctaccacgccatcatcggtgggatcgtggctttcattgtcttcctgctgctcatcatgctcatcttcctcggccactacttgatccggcacaaaggaacctacctgacacatgaggcaaaaggctccgacgatgctccagacgcggacacggccatcatcaatgcagaaggcgggcagtcaggaggggacgacaagaaggaatatttcatctagaggcgcctgcccacttcctgcgccccccaggggccctgtggggactgctggggccgtcaccaacccggacttgtacagagcaaccgcagggccgcccctcccgcttgctccccagcccacccacccccctgtacagaatgtctgctttgggtgcggttttgtactcggtttggaatggggagggaggagggcggggggaggggagggttgccctcagccctttccgtggcttctctgcatttgggttattattatttttgtaacaatcccaaatcaaatctgtctccaggctggagaggcaggagccctggggtgagaaaagcaaaaaacaaacaaaaaacaaaaccctggagtgttaggaggagagtgaaggtagaggggtgaggaagggtaaggggcagggctggtttcagctgggggctctcaccagccctcctttcagcctctacaacagagcagcttcccagacttctccaggaacccagaaacgggatggttgtcggcaaaggttgggagtggcttttcctctggtagccacacacctgagcactacggacagggaggcaggtgccaccttgacacctctcttccatagcaatgggaaagtgatgagtgcgggagtcctgaggagatgtggcctgcagacaacatgcagccatgcagggacccaggactgtaacctggggaggacgcgggtccctgcaaggaagagtagatttggagaggaaggatggaggtggactctcaccccattccccccggaaatgaacaaagccgggccctttccataggaactgcccttggagatagcagagtgtggctgcccctccttgctccagcagcagtgggagaggcactgctctggggcctgaactgcctctgcttccccccctgaggggcccctcactcttacccaagactctggattgttgcacggcaaccactcctcccatggcattgctcagcaactacttctcccttcccggccaccctgtgcccccttcctggtcccaacgccagcccttcatccttcctccctcagcagccaggcagacataacaacaaaactactaaaaggagcttcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - protein, 398 aa, Homo sapiens
MGAPAASLLLLLLLFACCWAPGGANLSQDDSQPWTSDETVVAGGTVVLKCQVKDHEDSSLQWSNPAQQTLYFGEKRALRDNRIQLVTSTPHELSISISNVALADEGEYTCSIFTMPVRTAKSLVTVLGIPQKPIITGYKSSLREKDTATLNCQSSGSKPAARLTWRKGDQELHGEPTRIQEDPNGKTFTVSSSVTFQVTREDDGASIVCSVNHESLKGADRSTSQRIEVLYTPTAMIRPDPPHPREGQKLLLHCEGRGNPVPQQYLWEKEGSVPPLKMTQESALIFPFLNKSDSGTYGCTATSNMGSYKAYYTLNVNDPSPVPSSSSTYHAIIGGIVAFIVFLLLIMLIFLGHYLIRHKGTYLTHEAKGSDDAPDADTAIINAEGGQSGGDDKKEYFI

SEQ ID NO: 5 - protein, 481 aa, Homo sapiens [sequence omitted]

SEQ ID NO: 6 - protein, 555 aa, Homo sapiens [sequence omitted]

(19) United States
(12) Patent Application Publication
(10) Pub. No.: US 2006/0233819 A1
(43) Pub. Date: Oct. 19, 2006

(54) Inhibition of T cell activation by butyrophilin 4 or B7-L1

(75) Inventors: Clive R. Wood, Boston, MA (US); Andrew J. Long, Chelmsford, MA (US); Beatriz M. Carreno, Clayton, MO (US)

Correspondence Address: FOLEY HOAG, LLP;PATENT GROUP, (w/WYS), 155 SEAPORT BLVD., BOSTON, MA, 02210-2600, US

(73) Assignee: Wyeth, Cambridge, MA (US)

(21) Appl. No.: 11/477,982

(22) Filed: Jun. 29, 2006

Related U.S. Application Data
(62) Division of application No. 10/463,260, filed on Jun. 17, 2003.
(60) Provisional application No. 60/389,660, filed on Jun. 17, 2002.

(51) Int. Cl. A61K 39/00

(52) U.S. Cl. 424/185.1

(57) ABSTRACT

The invention provides methods for modulating an immune response comprising contacting an immune cell with an agent that modulates BTF4 or B7-L1 mediated signaling. BTF4 or B7-L1 mediated signaling may either be increased, to thereby downregulate the immune response, or alternatively may be decreased, to upregulate the immune response. Modulation may be either activation or inhibition, and contacting may occur in vivo or in vitro. Performance of this method in vivo is also potentially therapeutic for an individual who may benefit from the upregulation or downregulation of an immune response. Such modulation to downregulate the immune response serves as a therapeutic e.g., for an individual who has received an organ transplant, or has a condition such as an allergy, or an autoimmune disorder. Alternatively, modulation to upregulate the immune response serves as a therapeutic for an individual, e.g., who has a condition such as an immunosuppressive disorder or a tumor. Methods for identifying an agent that modulates BTF4 signaling or B7-L1 signaling is also provided.

### RELATED APPLICATIONS

This application is a divisional of U.S. patent application Ser. No. 10/463,260 filed Jun. 17, 2003, which claims priority to U.S. provisional application Ser. No. 60/389,660, filed on Jun. 17, 2002; each application is incorporated herein in its entirety by this reference.

### BACKGROUND OF THE INVENTION

In order for T cells to respond to foreign proteins, two signals must be provided by antigen-presenting cells (APCs) to resting T lymphocytes (Jenkins, M. and Schwartz, R. (1987) J. Exp. Med. 165, 302-319; Mueller, D. L., et al. (1990) J. Immunol. 144, 3701-3709). The first signal, which confers specificity to the immune response, is transduced via the T cell receptor following recognition of foreign antigenic peptide presented in the context of the major histocompatibility complex (MHC). The second signal, termed costimulation, induces T cells to proliferate and become functional (Lenschow et al. 1996. Annu. Rev. Immunol. 14:233).). If T cells are only stimulated through the T cell receptor, without receiving an additional costimulatory signal, they become nonresponsive, anergic, or die, resulting in downmodulation of the immune response. Costimulation is neither antigen-specific, nor MHC restricted and is thought to be provided by one or more distinct cell surface molecules expressed by APCs (Jenkins, M. K., et al. 1988 J. Immunol. 140, 3324-3330; Linsley, P. S., et al. 1991 J. Exp. Med. 173, 721-730; Gimmi, C. D., et al., 1991 Proc. Natl. Acad. Sci. USA. 88, 6575-6579; Young, J. W., et al. 1992 J. Clin. Invest. 90, 229-237; Koulova, L., et al. 1991 J. Exp. Med. 173, 759-762; Reiser, H., et al. 1992 Proc. Natl. Acad. Sci. USA. 89, 271-275; van-Seventer, G. A., et al. (1990) J. Immunol. 144, 4579-4586; LaSalle, J. M., et al., 1991 J. Immunol. 147, 774-80; Dustin, M. I., et al., 1989 J. Exp. Med. 169, 503; Armitage, R. J., et al. 1992 Nature 357, 80-82; Liu, Y., et al. 1992 J. Exp. Med. 175, 437-445).

Members of the B7 family of proteins, B7-1 (CD80) and B7-2 (CD86), expressed on APCs are critical costimulatory molecules (Freeman et al. 1991. J. Exp. Med. 174:625; Freeman et al. 1989 J. Immunol. 143:2714; Azuma et al. 1993 Nature 366:76; Freeman et al. 1993. Science 262:909). B7 appears to play a predominant role during primary immune responses, while B7-1, which is upregulated later in the course of an immune response, may be important in prolonging primary T cell responses or costimulating secondary T cell responses (Bluestone. 1995. Immunity. 2:555).

Members of the B7 family of molecules such as B7-1 and B7-2, are known to bind the CD28 molecule. CD28 is a costimulatory receptor molecule which is constitutively expressed on resting T cells and increases in expression after activation. After signaling through the T cell receptor, ligation of CD28 and transduction of a costimulatory signal induces T cells to proliferate and secrete IL-2 (Linsley, P. S., et al. 1991 J. Exp. Med. 173, 721-730; Gimmi, C. D., et al. 1991 Proc. Natl. Acad. Sci. USA. 88, 6575-6579; June, C. H., et al. 1990 Immunol. Today. 11, 211-6; Harding, F. A., et al. 1992 Nature. 356, 607-609).

The receptor to which a B7 molecule binds (i.e., a costimulatory receptor or an inhibitory receptor), dictates whether the resulting signal to the immune cell is costimulation or inhibition. Several B7 family members exhibit binding affinity for both the costimulatory receptor CD28 and the inhibitory receptor CTLA4 (CD152). CTLA4 is not expressed on resting T cells and only appears following T cell activation (Brunet, J. F., et al., 1987 Nature 328, 267-270). CTLA4 appears to be critical in negative regulation of T cell responses (Waterhouse et al. 1995. Science 270:985). Blockade of CTLA4 has been found to remove inhibitory signals, while aggregation of CTLA4 has been found to provide inhibitory signals that downregulate T cell responses (Allison and Krummel. 1995. Science 270:932). The different expression patterns of the two receptors through the course of T cell activation is thought important for appropriate regulation of the T cell response, since the B7 molecules have a higher affinity for CTLA4 than for CD28 (Linsley, P. S., et al., 1991 J. Exp. Med. 174, 561-569). Members of the B7 family, such as B7-1 and B7-2, have been found to bind to distinct regions of the CTLA4 molecule and have different kinetics of binding to CTLA4 (Linsley et al. 1994. Immunity. 1:793).

Several additional B7 family members, B7-H1 (Dong, H. et al. (1999) Nat. Med. 5:1365-1369), ICOS-L (also known as GL50, B7h, LICOS, and B7RP-1) (Ling, V. et al. (2000) J. Immunol. 164:1653-7; Swallow, M. M. et al. (1999) Immunity 11:423-432; Aicher, A. et al. (2000) J. Immunol. 164:4689-96; Mages, H. W. et al. (2000) Eur. J. Immunol. 30:1040-7; Brodie, D. et al. (2000) Curr. Biol. 10:333-6; Yoshinaga, S. K. et al. (1999) Nature 402:827-32), B7-L1 (WO 98/44113; WO 00/53753; WO 00/08057; WO 00/08158; WO 00/32633; WO 98/54963; CN 1242376), and BTF4 (Tazi-Ahnini et al., Immunogenetics 47: 55-63 (1997); WO 98/33912; WO 99/07840) have recently been identified. B7-H1 (also known as PD-L1) exhibits immunoinhibitory rather than costimulatory activity in a T cell activation assay, and is known to interact with the immunoinhibitory receptor PD-1 (Freeman, G. J. et al. (2000) J. Exp. Med. 192:1027-34). PD-1 is expressed on activated T cells, B cells, and myeloid cells. ICOS-L exhibits costimulatory activity, and is known to interact with ICOS (Hutloff et al. (1999) Nature 397:263; WO 98/38216; Tamatani, T. et al. (2000) Int. Immunol. 12:51-55), a cell surface receptor which is related to CD28 and CTLA4. LDCAM is widely expressed in the non-immune tissue types, and is expressed on several immune cell types, including macrophages, and dendritic cells (WO 00/08158). B7-L1 may have other as yet unidentified binding partners. A binding partner for BTF4 has yet to be identified.

The importance of the B7:CD28/CTLA4 costimulatory/inhibitory pathway has been demonstrated in vitro and in several in vivo model systems. Blockade of this costimulatory pathway results in the development of antigen specific tolerance in murine and human systems (Harding, F. A., et al. (1992) Nature. 356, 607-609; Lenschow, D. J., et al. (1992) Science. 257, 789-792; Turka, L. A., et al. (1992) Proc. Natl. Acad. Sci. USA. 89, 11102-11105; Gimmi, C. D., et al. (1993) Proc. Natl. Acad. Sci USA 90, 6586-6590; Boussiotis, V., et al. (1993) J. Exp. Med. 178, 1753-1763). Conversely, expression of B7 by B7 negative murine tumor cells induces T-cell mediated specific immunity accompanied by tumor rejection and long lasting protection to tumor challenge (Chen, L., et al. (1992) Cell 71, 1093-1102; Townsend, S. E. and Allison, J. P. (1993) Science 259, 368-370; Baskar, S., et al. (1993) Proc. Natl. Acad. Sci. 90, 5687-5690.). Elucidation and manipulation of the costimulatory and immunoinhibitory pathways of the various B7 family members offers great potential to therapeutically manipulate immune response.

### SUMMARY OF THE INVENTION

The instant invention advances the prior art, inter alia, by providing methods of modulating signaling via BTF4 and/or B7-L1 to thereby modulate an immune response. Results of experiments detailed in the Examples section below indicate that both BTF4 and B7-L1 downregulate T cell receptor mediated activation of T cells. Accordingly, BTF4 and/or B7-L1 mediated signaling in an immune cell can be manipulated to modulate, either negatively or positively, antigen mediated activation of that cell. Modulation of the response of a particular immune cell, in turn, can lead to broader modulation of an immune response mounted by the various immune cell types which typically contribute to an overall immune response generated in an organism.

One aspect of the present invention relates to a fusion protein comprising the extracellular domain of a BTF4 molecule.

In one embodiment, the fusion protein comprises the amino acid sequence shown in SEQ ID NO:5.

Another aspect of the present invention relates to a fusion protein comprising the extracellular domain of a B7-L1 molecule.

In one embodiment, the fusion protein comprises the amino acid sequence shown in SEQ ID NO:6

Another aspect of the present invention relates to a method for downmodulating an immune response. The method comprises contacting an immune cell with an agent that increases BTF4 mediated signaling, to thereby downmodulate the immune response.

In one embodiment, the immune cell is a T cell.

In another embodiment, the agent is a BTF4 polypeptide.

In another embodiment, the agent is an extracellular portion of a BTF4 polypeptide crosslinked to an insoluble matrix.

In another embodiment, the agent is a BTF4-Ig fusion protein comprising the amino acid sequence shown in SEQ ID NO: 5, crosslinked to an insoluble matrix.

In one embodiment, the step of contacting occurs in vivo.

In another embodiment, the step of contacting occurs in vitro.

In one embodiment, the method further comprises contacting the T cell with an additional agent which downregulates an immune response.

Another aspect of the present invention relates to a method for upmodulating an immune response. The method comprises contacting an immune cell with an agent that decreases BTF4 mediated signaling, to thereby upmodulate the immune response.

In one embodiment, the agent is an antibody which specifically binds BTF4.

In one embodiment, the step of contacting occurs in vivo.

In another embodiment, the step of contacting occurs in vitro.

In one embodiment, the method further comprises contacting the T cell with an additional agent which upregulates an immune response.

Another aspect of the present invention relates to a method for treating a subject having a condition that would benefit from downregulation of an immune response. The method comprises administering to the subject an agent that promotes BTF4-mediated signaling such that a condition that would benefit from downregulation of an immune response is treated.

In one embodiment, the subject has a condition which is a transplant, an allergy, or an autoimmune disorder.

In another embodiment, the agent is a BTF4 polypeptide.

In one embodiment, the method further comprises administering a second agent which downregulates an immune response to the subject.

Another aspect of the present invention relates to a method for treating a subject having a condition that would benefit from upregulation of an immune response. The method comprises administering to the subject an agent that inhibits BTF4-mediated signaling such that the condition is treated.

In one embodiment, the condition is a tumor or an immunosuppressive disease.

In another embodiment, the agent is an antibody which specifically binds BTF4.

In one embodiment, the method further comprises administering to the subject, a second agent which upregulates an immune response.

Another aspect of the present invention relates to a method for identifying an agent that modulates BTF4 signaling. The method comprises providing a T cell, an appropriate activating signal, and a test agent, and assaying for modulation of BTF4 signaling by measuring a change in BTF4 modulation of T cell activation by the activating signal in the presence and absence of the test agent. A comparative change in modulation of T cell activation by BTF4 in the presence of the test agent indicates that the test agent is a modulator of BTF4 signaling.

Another aspect of the present invention relates to a method for downmodulating an immune response. The method comprises contacting an immune cell with an agent that increases B7-L1 mediated signaling, to thereby downmodulate the immune response.

In one embodiment, the immune cell is a T cell.

In another embodiment, the agent is a B7-L1 polypeptide.

In another embodiment, the agent is an extracellular portion of a B7-L1 polypeptide crosslinked to an insoluble matrix.

In another embodiment, the agent is a B7-L1-Ig fusion protein comprising the amino acid sequence shown in SEQ ID NO: 6, crosslinked to an insoluble matrix.

In one embodiment, the step of contacting occurs in vivo.

In another embodiment, the step of contacting occurs in vitro.

In one embodiment, the method further comprises contacting the T cell with an additional agent which downregulates an immune response.

Another aspect of the present invention relates to a method for upmodulating an immune response. The method comprises contacting an immune cell with an agent that decreases B7-L1 mediated signaling, to thereby upmodulate the immune response.

In one embodiment, the agent is an antibody which specifically binds B7-L1.

In one embodiment, the step of contacting occurs in vivo.

In another embodiment, the step of contacting occurs in vitro.

In one embodiment, the method further comprises contacting the T cell with an additional agent which upregulates an immune response.

Another aspect of the present invention relates to a method for treating a subject having a condition that would benefit from downregulation of an immune response. The method comprises administering to the subject an agent that promotes B7-L1-mediated signaling such that a condition that would benefit from downregulation of an immune response is treated.

In one embodiment, the condition is transplant, an allergy, or an autoimmune disorder.

In another embodiment, the agent is a B7-L1 polypeptide.

In one embodiment, the method further comprises administering to the subject a second agent which downregulates an immune response.

Another aspect of the present invention relates to a method for treating a subject having a condition that would benefit from upregulation of an immune response. The method comprises administering to the subject an agent that inhibits B7-L1-mediated signaling such that a condition that would benefit from upregulation of an immune response is treated.

In one embodiment the condition is a tumor or an immunosuppressive disease.

In one embodiment, the agent is an antibody which specifically binds B7-L1.

In another embodiment, the method further comprises administering to the subject a second agent which upregulates an immune response.

Another aspect of the present invention also relates to a method for identifying an agent that modulates B7-L1 signaling. The method comprises providing a T cell, an appropriate activating signal, and a test agent, and then assaying for modulation of B7-L1 signaling by measuring a change in B7-L1 modulation of T cell activation by the activating signal in the presence and absence of the test agent. A comparative change in modulation of T cell activation by B7-L1 in the presence of the test agent indicates that the test agent is an modulator of B7-L1 signaling.

### BRIEF DESCRIPTION OF THE DRAWINGS

FIG. 1 contains 4 bar graphs, representing the results of T cell activation assays. T cells were activated in the presence and absence of BTF4 or B7-L1, or the costimulatory molecules ICOS-L or B7-2, or the known T cell inhibitor PD-L1. The bottom graphs are duplicate experiments of the top graphs.

### DETAILED DESCRIPTION OF THE INVENTION

BTF4 and B7-L1 are cell surface molecules. Without being bound by theory, when an immune cell, such as a T cell, contacts a cell which expresses BTF4 or B7-L1, the BTF4 or B7-L1 molecule binds to a specific binding partner (also referred to in the art as a receptor or a counterstructure) present on the immune cell, (e.g., a T cell) which then transmits a signal that modulates the activation state of the immune cell. As demonstrated herein, BTF4 and B7-L1 downregulate activation of a cell by binding receptors which function as immunoinhibitory receptors on the cell. Accordingly, the activation state of an immune cell can be modulated by manipulation of BTF4 and B7-L1 signaling. For example, in one embodiment, an agent is used to decrease BTF4 or B7-L1 mediated signaling, to thereby upregulate the immune response. This may be achieved, for instance, by contacting the cells with an agent (e.g., an antibody or a small molecule) that inhibits the interaction of BTF4 or B7-L1 with their receptor. In another embodiment, an agent is used to increase BTF4 or B7-L1 mediated signaling, to thereby downregulate an immune response. For example, BTF4 or B7-L1 mediated signaling can be increased by increasing the amount of BTF4 or B7-L1 binding to receptors on an immune cell to activate signaling.

One of skill in the art will recognize that modification of BTF4 or B7-L1 mediated signaling can be achieved by manipulation of the immune cell expressing the BTF4 or B7-L1 receptor, or of the cell expressing BTF4 or B7-L1, or both.

In one embodiment, the interaction of BTF4 or B7-L1 expressed on an antigen presenting cell with an immunoinhibitory receptor expressed on an immune cell (e.g., a T cell or B cell) is modulated to produce the desired effect. The interaction is modulated, for instance by contacting the antigen presenting cell or the immune cell with a modulator of BTF4 or B7-L1 mediated signaling that functions by either enhancing or inhibiting BTF4 or B7-L1 receptor binding, e.g., inhibit binding by the addition of a blocking antibody, enhance binding by adding an activating form of BTF4 or B7-L1.

Another way to modulate BTF4 or B7-L1 signaling is to modulate expression of the protein in the cell (e.g., an antigen presenting cell) which contacts the immune cell. Expression of BTF4 or B7-L1 in a cell can be increased by expression of an exogenous nucleic acid molecule encoding BTF4 or B7-L1 polypeptide that has been introduced into the cell. Alternatively the cell can be contacted with an agent which increases expression of endogenous BTF4 or B7-L1.

I. Definitions

As used herein, the term “immune cell” includes cells that are of hematopoietic origin and that play a role in the immune response. Immune cells include lymphocytes, such as B cells and T cells; natural killer cells; myeloid cells, such as monocytes, macrophages, eosinophils, mast cells, basophils, and granulocytes.

As used herein, the term “T cell” includes CD4⁺ T cells and CD8+ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells) as well as other antigen presenting cells (e.g., keratinocytes, endothelial cells, astrocytes, fibroblasts, oligodendrocytes).

As used herein, the term “immune response” includes T cell mediated and/or B cell mediated immune responses that are influenced by modulation of T cell costimulation. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

As used herein, the term “costimulatory receptor” includes receptors which transmit a costimulatory signal to a immune cell, e.g., CD28. With respect to T cells, transmission of a costimulatory signal to a T cell involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death in the T cell. As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. Immune cells that have received a cell-receptor mediated signal, e.g., via an activating receptor are referred to herein as “activated immune cells.”

As used herein, the term “inhibitory receptors” or “immunoinhibitory receptors” includes receptors which transmit an inhibitory signal to an immune cell (e.g., CTLA4 or PD-1). An inhibitory signal as transduced by an inhibitory receptor can occur even if a costimulatory receptor (such as CD28) is not present on the immune cell and, thus, is not simply a function of competition between inhibitory receptors and costimulatory receptors for binding of costimulatory molecules (Fallarino et al. 1998. J. Exp. Med. 188:205). Transmission of an inhibitory signal to an immune cell can result in unresponsiveness or anergy or programmed cell death in the immune cell.

As used herein, the term “BTF4 receptor” refers to a receptor which binds to a BTF4 molecule and transmits a signal to an immune cell. In one embodiment, the binding of a BTF4 molecule expressed on the surface of a cell transmits a negative signal to an immune cell upon binding to a BTF4 receptor. The term “B7-L1 receptor” refers to a receptor which binds to a B7-L1 molecule and transmits a signal to an immune cell. In one embodiment, the binding of a B7-L1 molecule expressed on the surface of a cell transmits a negative signal to an immune cell upon binding to a B7-L1 receptor. In one embodiment, a B7-L1 receptor is LDCAM

As used herein, the term “inhibitory signal” refers to a signal transmitted via an inhibitory receptor (e.g., CTLA4, PD-1, BTF4 receptor, or B7-L1 receptor) on a immune cell. Such a signal antagonizes a signal transmitted via an activating receptor (e.g., via a T cell receptor, CD3, B cell receptor, or Fc molecule) and can result, e.g., in: inhibition of second messenger generation; an inhibition of proliferation; an inhibition of effector function in the immune cell, e.g., reduced phagocytosis, reduced antibody production, reduced cellular cytotoxicity, the failure of the immune cell to produce mediators, (such as cytokines (e.g., IL-2) and/or mediators of allergic responses); or the development of anergy.

As used herein, the term “activating receptor” includes immune cell receptors that bind antigen, complexed antigen (e.g., in the context of MHC molecules), or bind to antibodies. Such activating receptors include T cell receptors, B cell receptors, cytokine receptors, LPS receptors, complement receptors, and Fc receptors.

For example, T cell receptors are present on T cells and are associated with CD3 molecules. T cell receptors are stimulated by antigen in the context of MHC molecules (as well as by polyclonal T cell activating reagents). T cell activation via the T cell receptor results in numerous changes, e.g., protein phosphorylation, membrane lipid changes, ion fluxes, cyclic nucleotide alterations, RNA transcription changes, protein synthesis changes, and cell volume changes.

B cell receptors are present on B cells. B cell antigen receptors are a complex between membrane Ig (mIg) and other transmembrane polypeptides (e.g., Igα and Igβ). The signal transduction function of mIg is triggered by crosslinking of receptor molecules by oligomeric or multimeric antigens. B cells can also be activated by anti-immunoglobulin antibodies. Upon B cell receptor activation, numerous changes occur in B cells, including tyrosine phosphorylation.

Fc receptors are found on many cells which participate in immune responses. Fc receptors (FcRs) are cell surface receptors for the Fc portion of immunoglobulin molecules (Igs). Among the human FcRs that have been identified so far are those which recognize IgG (designated Fcγ R), IgE (Fcε R1), IgA (Fcα), and polymerized IgM/A (Fcμα R). FcRs are found in the following cell types: Fcε R I (mast cells), Fcε R.II (many leukocytes), Fcα R (neutrophils), and Fcμα R (glandular epithelium, hepatocytes) (Hogg, N., 1988, Immun. Today, 9:185-86). The widely studied FcγRs are central in cellular immune defenses, and are responsible for stimulating the release of mediators of inflammation and hydrolytic enzymes involved in the pathogenesis of autoimmune disease (Unkeless, J. C., 1988, Ann. Rev. Imm., 6:25 1-87). The FcγRs provide a crucial link between effector cells and the lymphocytes that secrete Ig, since the macrophage/monocyte, polymorphonuclear leukocyte, and natural killer (NK) cell FcγRs confer an element of specific recognition mediated by IgG. Human leukocytes have at least three different receptors for IgG: h Fcγ RI (found on monocytes/macrophages), hFcγ RII (on monocytes, neutrophils, eosinophils, platelets, possibly B cells, and the K562 cell line), and Fcγ RIII (on NK cells, neutrophils, eosinophils, and macrophages).

As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and, thus, failure to proliferate. Anergic T cells can, however, mount responses to unrelated antigens and can proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. 1992. Science. 257:1134).

BTF4 or B7-L1 mediated signaling is defined herein as one or more cellular events directly or indirectly induced in an immune cell which possess receptors for BTF4 or B7-L1 when the receptor is triggered, e.g., by the binding of BTF4 or B7-L1 to the receptor. Triggering of the receptor initiates a signaling event which results in a cellular change. Such a cellular event can be detected, for instance, by measuring a change in the activation state of the immune cell, e.g., a change in second messenger generation, expression of activation markers, or effector function. For example, in one embodiment, T cell activation can be measured, in experiments similar to those detailed in the Examples section below. BTF4 or B7-L1 signaling can be modulated, e.g., by modulating the expression and/or activity of BTF4 or B7-L1.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

II. Modulators of BTF4 and B7-L1 Mediated Signaling

Various agents can be used to modulate BTF4 or B7-L1 mediated signaling. For example, an agent which binds to the BTF4 or B7-L1 receptor and intitiates signaling via the receptor (e.g., by crosslinking the receptor) may be used as an agent for increasing BTF4 or B7-L1 mediated signaling. Several examples of such agents are provided and described in detail below. Conversely, an agent which binds to the BTF4 or B7-L1 receptor but does not activate the receptor can be used as an agent for decreasing BTF4 or B7-L1 mediated signaling, e.g., by competing with BTF4 or B7-L1 for binding or by blocking BTF4 or B7-L1 binding. Several examples of such agents are also provided and described in detail below.

A. Isolated BTF4 or B7-L1 Nucleic Acid Molecules

In one embodiment, a modulatory agent useful for modulating the activity and/or expression of BTF4 or B7-L1 mediated signaling comprises an isolated nucleic acid molecule that encodes BTF4 or B7-L1 protein or biologically active portion thereof. Preferably the nucleic acid molecule encodes eukaryotic BTF4 or B7-L1, and more preferably human BTF4 or B7-L1. In one embodiment, a BTF4 or B7-L1 molecule has the sequence shown in SEQ ID NO: 1 (BTF4) or SEQ ID NO: 3 (B7-L1).

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid) in the genomic DNA of the organism from which the nucleic acid molecule is derived. For example, in various embodiments, the isolated BTF4 or B7-L1 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. An “isolated” BTF4 or B7-L1 nucleic acid molecule may, however, be linked to other nucleotide sequences that do not normally flank the BTF4 or B7-L1 sequences in genomic DNA (e.g., the BTF4 or B7-L1 nucleotide sequences may be linked to vector sequences). In certain preferred embodiments, an “isolated” nucleic acid molecule, such as a cDNA molecule, also may be free of other cellular material. However, it is not necessary for the BTF4 or B7-L1 nucleic acid molecule to be free of other cellular material to be considered “isolated” (e.g., a BTF4 or B7-L1 DNA molecule separated from other mammalian DNA and inserted into a bacterial cell would still be considered to be “isolated”).

A nucleic acid molecule of the present invention, e.g., a nucleic acid molecule having the nucleotide sequence of SEQ ID NO: 1 (BTF4) or SEQ ID NO: 3 (B7-L1), or a portion thereof, can be isolated using standard molecular biology techniques and the sequence information provided herein. For example, using all or portion of the nucleic acid sequence of SEQ ID NO: 1 or 3 as a hybridization probe, BTF4 or B7-L1 nucleic acid molecules can be isolated using standard hybridization and cloning techniques (e.g., as described in Sambrook, J. et al. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO: 1 or 3 can be isolated by the polymerase chain reaction (PCR) using synthetic oligonucleotide primers designed based upon the sequence of SEQ ID NO: 1 or 3, respectively.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to BTF4 or B7-L1 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO: 1 or 3.

In another preferred embodiment, an isolated nucleic acid molecule of the invention comprises a nucleic acid molecule which is a complement of the nucleotide sequence shown in SEQ ID NO: 1 or 3, or a portion of any of these nucleotide sequences. A nucleic acid molecule which is complementary to the nucleotide sequence shown in SEQ ID NO: 1 or 3, is one which is sufficiently complementary to the nucleotide sequence shown in SEQ ID NO: 1 or 3, respectively, such that it can hybridize to the nucleotide sequence shown in SEQ ID NO: 1 or 3, respectively, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 95%, 98% or more homologous to the nucleotide sequence (e.g., to the entire length of the nucleotide sequence) shown in SEQ ID NO: 1 or 3 or a portion of any of these nucleotide sequences.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which need to be introduced for optimal alignment of the two sequences. As used herein amino acid or nucleic acid “identity” is equivalent to amino acid or nucleic acid “homology”.

The comparison of sequences and determination of percent identity between two sequences can be accomplished using a mathematical algorithm. In a preferred embodiment, the percent identity between two amino acid sequences is determined using the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package using a NWSgapdna.CMP matrix and a gap weight of 40, 50, 60, 70, or 80 and a length weight of 1, 2, 3, 4, 5, or 6.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to BTF4 or B7-L1 nucleic acid molecules of the invention. BLAST protein searches can be performed with the XBLAST program, score=50, wordlength=3 to obtain amino acid sequences homologous to BTF4 or B7-L1 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. For example, the nucleotide sequences of the invention were analyzed using the default Blastn matrix 1-3 with gap penalties set at: existence 11 and extension 1. The amino acid sequences of the invention were analyzed using the default settings: the Blosum 62 matrix with gap penalties set at existence 11 and extension 1. See, e.g, the NIH web page.

Moreover, the nucleic acid molecule of the invention can comprise only a portion of the nucleic acid sequence of SEQ ID NO: 1 or 3, for example a fragment encoding a biologically active portion (e.g., a receptor binding portion) of a BTF4 or B7-L1 protein. In one embodiment, a biologically active portion of a BTF4 or B7-L1 protein comprises the extracellular domain of the naturally occurring protein. One example of this is the portion corresponding to from about amino acid 1 to about amino acid 249 of SEQ ID NO: 2 or from about amino acid 1 to about amino acid 323 of SEQ ID NO: 4. In a preferred embodiment, the extracellular domain comprises from amino acid 1 to amino acid 249 of SEQ ID NO: 2 or from amino acid 1 to amino acid 323 of SEQ ID NO: 4.

At least two naturally occuring forms of B7-L1 protein are known in the art. One is 398 in length (set forth in SEQ ID NO: 4) and one is 432 a.a. (WO 00/08057; WO 00/08158; WO 00/32633). These two forms differ after position 29, at which point the long form contains a 34 a.a. insert. After the insert, the two forms are identical in sequence. In one embodiment of the invention, the nucleic acid molecule of the invention comprises a biologically active portion (e.g., a receptor binding portion) of the long form of B7-L1. In another embodiment, the nucleic acid moleucle of the invention comprises a biologically active portion (e.g., a receptor binding portion) of the short form of B7-L1. In one embodiment, a biologically active portion of the B7-L1 protein comprises the extracellular domain of the naturally occurring protein.

A nucleic acid fragment encoding a “biologically active portion of a BTF4 or B7-L1 protein” can be prepared by isolating a portion of the nucleotide sequence of SEQ ID NO:1 or 3, or other nucleotide sequence which encodes a polypeptide having the respective BTF4 or B7-L1 biological activity (the biological activities of the BTF4 or B7-L1 proteins are described herein), expressing the encoded portion of the BTF4 or B7-L1 protein (e.g., by recombinant expression in vitro) and assessing the activity of the encoded portion of the BTF4 or B7-L1 protein.

Nucleic acid molecules, that differ from SEQ ID NO: 1 or 3 due to degeneracy of the genetic code, and thus encode the same BTF4 or B7-L1 protein as that encoded by SEQ ID NO: 1 or 2, are also encompassed by the invention. Accordingly, in another embodiment, an isolated nucleic acid molecule of the invention has a nucleotide sequence encoding a protein having an amino acid sequence shown in SEQ ID NO: 1 or 3, or a biologically active portion thereof, as described herein. In another embodiment, an isolated nucleic acid molecule of the invention has a nucleotide sequence encoding a BTF4 or B7-L1 protein.

Moreover, nucleic acid molecules encoding other BTF4 or B7-L1 family members and, thus, which have a nucleotide sequence which differs from the BTF4 or B7-L1 family sequences of SEQ ID NO: 1 or 3, are intended to be within the scope of the invention. For example, another BTF4 or B7-L1 cDNA can be identified based on the nucleotide sequence of human BTF4 or B7-L1. As such, biologically active portions of other BTF4 or B7-L1 family members (e.g., which correspond to the receptor binding region and/or extracellular portions) are also within the scope of the present invention. Moreover, nucleic acid molecules encoding BTF4 or B7-L1 proteins from different species, and thus which have a nucleotide sequence which differs from the BTF4 or B7-L1 sequences of SEQ ID NO: 1 or 3 are intended to be within the scope of the invention. For example, a mouse BTF4 or B7-L1 cDNA can be identified based on the nucleotide sequence of a human BTF4 or B7-L1 molecule. As such, biologically active portions of other BTF4 or B7-L1 species (e.g., which correspond to the receptor binding region and/or extracellular portions) are also within the scope of the present invention.

In addition, biologically active portions, as described above, of natural allelic variants and homologs of the BTF4 or B7-L1 genes or cDNAs are also intended to be included within the scope of the invention. Nucleic acid molecules corresponding to natural allelic variants and homologues of the BTF4 or B7-L1 cDNAs of the invention can be isolated based on their homology to the BTF4 or B7-L1 nucleic acids disclosed herein using the cDNAs disclosed herein, or a portion thereof, as a hybridization probe according to standard hybridization techniques. In one embodiment, a nucleic acid molecule encoding BTF4 or B7-L1 hybridizes over its full length to a nucleic acid molecule comprising a nucleotide sequence which is the complement of that shown in SEQ ID NO: 1 or SEQ ID NO:3. In another embodiment, a nucleic acid molecule encoding BTF4 or B7-L1 hybridizes to the portion of a nucleic acid molecule comprising a nucleotide sequence which is the complement of the portion of SEQ ID NO: 1 or SEQ ID NO:3 which encodes the extracellular domain of B7-L1 or BTF4.

For example, a BTF4 or B7-L1 DNA can be isolated from a human genomic DNA library using all or portion of SEQ ID NO: 1 or 3 as a hybridization probe and standard hybridization techniques (e.g., as described in Sambrook, J., et al. Molecular Cloning: A Laboratory Manual. 2nd, ed., Cold Spring Harbor Laboratory, Cold Spring Harbor, N.Y., 1989). Moreover, a nucleic acid molecule encompassing all or a portion of a BTF4 or B7-L1 gene can be isolated by the polymerase chain reaction using oligonucleotide primers designed based upon the sequence of SEQ ID NO: 1 or 3. For example, mRNA can be isolated from cells (e.g., by the guanidinium-thiocyanate extraction procedure of Chirgwin et al. (1979) Biochemistry 18:5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO: 1 or 3. A nucleic acid molecule of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a BTF4 or B7-L1 nucleotide sequence can be prepared by standard synthetic techniques, e.g., using an automated DNA synthesizer.

In another embodiment, an isolated nucleic acid molecule of the invention is at least 400 (e.g., 429), 450, 500, or 550, or more nucleotides in length and hybridizes under stringent conditions to the nucleic acid molecule comprising the nucleotide sequence of SEQ ID NO: 1 or 3, or a fragment thereof which encodes a biologically active portion thereof, as described above. In another embodiment, the nucleic acid molecule is at least 600, 650, 700, 750, 800, 850, 900 (e.g., 909), or 950, or more nucleotides in length.

As used herein, the term “hybridizes under stringent conditions” is intended to describe conditions for hybridization and washing under which nucleotide sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO: 1 or 3 corresponds to a naturally-occurring nucleic acid molecule.

As used herein, the terms “gene” and “recombinant gene” refer to nucleic acid molecules which include an open reading frame encoding a BTF4 or B7-L1 protein, preferably a mammalian BTF4 or B7-L1 protein, and can further include non-coding regulatory sequences, and introns.

As used herein, a “naturally-occurring” nucleic acid molecule refers to an RNA or DNA molecule having a nucleotide sequence that occurs in nature (e.g., encodes a natural protein). In addition to the BTF4 or B7-L1 nucleotide sequences shown in SEQ ID NO: 1 or 3, it should be appreciated by those skilled in the art that DNA sequence polymorphisms that lead to minor changes in the nucleotide or amino acid sequences of a BTF4 or B7-L1 may exist within a population (e.g., the human population). Such genetic polymorphism in a BTF4 or B7-L1 gene may exist among individuals within a population due to natural allelic variation. Such natural allelic variations include both functional and non-functional BTF4 or B7-L1 proteins, and can typically result in 1-5% variance in the nucleotide sequence of the gene. Such nucleotide variations and resulting amino acid polymorphisms in BTF4 or B7-L1 genes that are the result of natural allelic variation and that do not alter the functional activity of a BTF4 or B7-L1 polypeptide are within the scope of the invention. The nucleic acid which arises from a natural allelic variation is referred to as an allelic variant of the gene or message. The polypeptide which is encoded by such a nucleic acid is referred to as an allelic variant of the protein.

In addition to naturally-occurring allelic variants of BTF4 or B7-L1 sequences that may exist in the population, the skilled artisan will further appreciate that minor changes may be introduced by mutation into nucleotide sequences, e.g., of SEQ ID NO: 1 or 3, thereby leading to changes in the amino acid sequence of the encoded protein, without altering the functional activity of a BTF4 or B7-L1 protein. For example, nucleotide substitutions leading to amino acid substitutions at “non-essential” amino acid residues may be made in the sequence of SEQ ID NO: 1 or 3. A “non-essential” amino acid residue is a residue that can be altered from the wild-type sequence of a BTF4 or B7-L1 nucleic acid molecule (e.g., the sequence of SEQ ID NO: 1 or 3) without altering the functional activity of a BTF4 or B7-L1 molecule. Preferably, residues in the extracellular domain of BTF4 or B7-L1 which are found to be required for binding of BTF4 or B7-L1 to a receptor (e.g., identified using an alanine scanning mutagenesis screen or other art recognized screening assay) are not altered. Exemplary residues of BTF4 or B7-L1 which are non-essential and, therefore, amenable to substitution, can be identified by one of ordinary skill in the art by performing an amino acid alignment of B7 family members and determining residues that are not conserved. Such residues, because they have not been conserved, are more likely amenable to substitution.

Accordingly, another aspect of the invention pertains to nucleic acid molecules encoding BTF4 or B7-L1 proteins that contain changes in amino acid residues that are not essential for a BTF4 or B7-L1 activity. Such BTF4 or B7-L1 proteins differ in amino acid sequence from SEQ ID NO: 1 and 3 yet retain an inherent activity. An isolated nucleic acid molecule encoding a non-natural variant of a BTF4 or B7-L1 protein can be created by introducing one or more nucleotide substitutions, additions or deletions into the nucleotide sequence of SEQ ID NO: 1 or 3 such that one or more amino acid substitutions, additions or deletions are introduced into the encoded protein. Mutations can be introduced into SEQ ID NO: 1 or 3 by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a BTF4 or B7-L1 is preferably replaced with another amino acid residue from the same side chain family.

Alternatively, in another embodiment, mutations can be introduced randomly along all or part of a BTF4 or B7-L1 coding sequence, such as by saturation mutagenesis, and the resultant mutants can be screened for their ability to bind to DNA and/or activate transcription, to identify mutants that retain functional activity. Following mutagenesis, the encoded BTF4 or B7-L1 mutant protein can be expressed recombinantly in a host cell and the functional activity of the mutant protein can be determined using assays available in the art for assessing a BTF4 or B7-L1 activity.

Accordingly, another aspect of the invention pertains to nucleic acid molecules encoding BTF4 or B7-L1 proteins that contain changes in amino acid residues that are not essential for activity.

Yet another aspect of the invention pertains to isolated nucleic acid molecules encoding a BTF4 or B7-L1 fusion protein. Such nucleic acid molecules, comprising at least a first nucleotide sequence encoding a BTF4 or B7-L1 protein, polypeptide or peptide operatively linked to a second nucleotide sequence encoding a non-BTF4 or B7-L1 protein (heterologous), polypeptide or peptide, can be prepared by standard recombinant DNA techniques. BTF4 and B7-L1 fusion proteins are discussed further below.

In addition to the nucleic acid molecules which encode BTF4 or B7-L1 proteins described above, isolated nucleic acid molecules which are antisense thereto can be used as modulating agents to effect modulation of the BTF4 or B7-L1 mediated signaling. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire BTF4 or B7-L1 coding strand, or only to a portion thereof. In one embodiment, an antisense nucleic acid molecule is antisense to a “coding region” of the coding strand of a nucleotide sequence encoding BTF4 or B7-L1. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding BTF4 or B7-L1. The term “noncoding region” refers to 5′ and 3′ sequences which flank the coding region that are not translated into amino acids (i.e., also referred to as 5′ and 3′ untranslated regions).

Given the coding strand sequences encoding BTF4 or B7-L1 disclosed herein, antisense nucleic acids of the invention can be designed according to the rules of Watson and Crick base pairing. The antisense nucleic acid molecule can be complementary to the entire coding region of BTF4 or B7-L1 mRNA, but more preferably is an oligonucleotide which is antisense to only a portion of the coding or noncoding region of BTF4 or B7-L1 mRNA. For example, the antisense oligonucleotide can be complementary to the region surrounding the translation start site of BTF4 or B7-L1 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid is of an antisense orientation to a target nucleic acid of interest, described further in the following subsection).

The antisense nucleic acid molecules would inhibit expression of BTF4 or B7-L1, and thus inhibit BTF4 or B7-L1 mediated signaling. Such inhibition would result in upregulation of an immune response by an immune cell, and thus upregulation of an immune response in an individual to whom the antisense nucleic acid molecules were administered.

The antisense nucleic acid molecules of the invention are typically administered to a subject or generated in situ such that they hybridize with or bind to cellular mRNA and/or genomic DNA encoding a BTF4 or B7-L1 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid molecule is a ribozyme. Ribozymes are catalytic RNA molecules with ribonuclease activity which are capable of cleaving a single-stranded nucleic acid molecule, such as an mRNA, to which they have a complementary region. Thus, ribozymes (e.g., hammerhead ribozymes (described in Haseloff and Gerlach (1988) Nature 334:585-591)) can be used to catalytically cleave BTF4 or B7-L1 mRNA transcripts to thereby inhibit translation of BTF4 or B7-L1 mRNA. A ribozyme having specificity for a BTF4 or B7-L1-encoding nucleic acid can be designed based upon the nucleotide sequence of a BTF4 or B7-L1 cDNA disclosed herein (i.e., SEQ ID NO: 1 or 3). For example, a derivative of a Tetrahymena L-19 IVS RNA can be constructed in which the nucleotide sequence of the active site is complementary to the nucleotide sequence to be cleaved in a BTF4 or B7-L1-encoding mRNA. See, e.g., Cech et al. U.S. Pat. No. 4,987,071; and Cech et-al. U.S. Pat. No. 5,116,742. Alternatively, BTF4 or B7-L1 mRNA can be used to select a catalytic RNA having a specific ribonuclease activity from a pool of RNA molecules. See, e.g., Bartel, D. and Szostak, J. W. (1993) Science 261:1411-1418.

Alternatively, BTF4 or B7-L1 gene expression can be inhibited by targeting nucleotide sequences complementary to the regulatory region of the BTF4 or B7-L1 (e.g., the BTF4 or B7-L1 promoter and/or enhancers) to form triple helical structures that prevent transcription of the BTF4 or B7-L1 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioessays 14(12):807-15.

In another embodiment, a compound that promotes RNAi can be used to inhibit BTF4 or B7-L1 expression. RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In yet another embodiment, the BTF4 or B7-L1 nucleic acid molecules of the present invention can be modified at the base moiety, sugar moiety, or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

PNAs of BTF4 or B7-L1 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of BTF4 or B7-L1 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup and Nielsen (1996) supra)); or as probes or primers for DNA sequencing or hybridization (Hyrup B. and Nielsen (1996) supra; Perry-O'Keefe et al. (1996) supra).

In another embodiment, PNAs of BTF4 or B7-L1 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of BTF4 or B7-L1 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. and Nielsen (1996) supra). The synthesis of PNA-DNA chimeras can be performed as described in Hyrup B. and Nielsen (1996) supra and Finn P. J. et al. (1996) Nucleic Acids Res. 24(17):3357-63. For example, a DNA chain can be synthesized on a solid support using standard phosphoramidite coupling chemistry. Modified nucleoside analogs, (e.g., 5′-(4-methoxytrityl)amino-5′-deoxy-thymidine phosphoramidite), can be used as a linker between the PNA and the 5′ end of DNA (Mag, M. et al. (1989) Nucleic Acid Res. 17:5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Biotechniques 6:958-976) or intercalating agents. (See, e.g., Zon (1988) Pharm. Res. 5:539-549). To this end, the oligonucleotide can be conjugated to another molecule, (e.g., a peptide, hybridization triggered cross-linking agent, transport agent, or hybridization-triggered cleavage agent).

B. Isolated Polypeptides and Antibodies that Recognize BTF4 or B7-L1

Exemplary agents useful for increasing BTF4 or B7-L1 mediated signaling are BTF4 or B7-L1 polypeptides or biologically active portions thereof. BTF4 or B7-L1 have the ability to bind to the BTF4 receptor or B7-L1 receptor, and transmit a signal via the receptor. Generally speaking, activation of the receptor requires crosslinking of the receptor. Thus, preferably, the activating form of BTF4 or B7-L1 is multivalent. When contacted with a cell that expresses the BTF4 or B7-L1 receptor, the multivalent form binds to and crosslinks the receptor to transmit a signal via the receptor. An activating form of BTF4 or B7-L1 may be, e.g., soluble or may be a monovalent form of the molecule linked to a solid or semisolid substrate.

In more than one embodiment of the present invention, it may be desirable to immobilize the modulator of BTF4 or B7-L1 mediated signaling on the surface of an insoluble matrix. Examples of such solid or semi-solid matrixes include, without limitation, any number of substances, such as polypropylene, polystyrene, sepharose, and agarose, molded into a formed object such as microbeads or cell culture wells. Also included are a cell or cell-like (e.g., comprising a lipid bilayer) surface or any number of gel like matrixes molded into a formed object.

In one embodiment, a fusion protein can be provided which adds a domain that allows the protein to be bound to a matrix. For example, glutathione-S-transferase/BTF4 or B7-L1 fusion proteins or glutathione-S-transferase/target fusion proteins can be adsorbed onto glutathione sepharose beads (Sigma Chemical, St. Louis, Mo.) or glutathione derivatized microtiter plates.

Other techniques for immobilizing proteins on matrices can also be used in the present invention. For example, a BTF4 or B7-L1 can be immobilized utilizing conjugation of biotin and streptavidin. Biotinylated BTF4 or B7-L1 protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with BTF4 or B7-L1 protein or target molecules but which do not interfere with binding of the BTF4 or B7-L1 protein to its target molecule can be derivatized to the wells of the plate, and unbound target or BTF4 or B7-L1 protein trapped in the wells by antibody conjugation. Methods for detecting such complexes, in addition to those described above for the GST-immobilized complexes, include immunodetection of complexes using antibodies reactive with the BTF4 or B7-L1 protein, as well as enzyme-linked assays which rely on detecting an enzymatic activity associated with the BTF4 or B7-L1 protein or target molecule.

In one embodiment, native BTF4 or B7-L1 proteins can be isolated from cells or tissue sources by an appropriate purification scheme using standard protein purification techniques. In another embodiment, BTF4 or B7-L1 proteins are produced by recombinant DNA techniques. As an alternative to recombinant expression, a BTF4 or B7-L1 protein or polypeptide can be synthesized chemically using standard peptide synthesis techniques.

Other techniques for immobilizing proteins on matrices can also be used in the present invention. For example, a BTF4 or B7-L1 can be immobilized utilizing conjugation of biotin and streptavidin. Biotinylated BTF4 or B7-L1 protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with BTF4 or B7-L1 protein or target molecules but which do not interfere with binding of the BTF4 or B7-L1 protein to its target molecule can be derivatized to the wells of the plate, and unbound target or BTF4 or B7-L1 protein trapped in the wells by antibody conjugation. Methods for detecting such complexes, in addition to those described above for the GST-immobilized complexes, include immunodetection of complexes using antibodies reactive with the BTF4 or B7-L1 protein, as well as enzyme-linked assays which rely on detecting an enzymatic activity associated with the BTF4 or B7-L1 protein or target molecule.

In one embodiment, the isolated BTF4 or B7-L1 proteins comprise the amino acid sequence encoded by SEQ ID NO: 2 or 4, respectively, or biologically active fragments thereof. In another preferred embodiment, the protein comprises the amino acid sequence of amino acid 1-249, of SEQ ID NO: 2, which is the extracellular portion of the BTF4 molecule, or comprises the amino acid sequence of amino acid 1-357, of SEQ ID NO: 4, which is the extracellular portion of the B7-L1 molecule. In other embodiments, the protein has at least 50%, at least 60% amino acid identity, more preferably 70% amino acid identity, more preferably 80%, and even more preferably, 90% or 95% amino acid identity with the amino acid sequence shown in SEQ ID NO: 2 or SEQ ID NO:4 or amino acids 1-143, of SEQ ID NO: 2, or amino acids 1-357, of SEQ ID NO: 4.

As discussed above, the invention further pertains to soluble forms of BTF4 or B7-L1 proteins. Such forms can be naturally occurring, e.g., as shown in SEQ ID NO:2 or can be engineered and can comprise or consist of, e.g., an extracellular domain of a BTF4 or B7-L1 protein. In one embodiment, the extracellular domain of the BTF4 or B7-L1 polypeptide comprises the mature form of a BTF4 or B7-L1 polypeptide, but not the transmembrane and cytoplasmic domains. A soluble form of BTF4 or B7-L1, or a receptor binding portion thereof, which is multivalent to the extent that it is sufficient to crosslink the receptor is also considered an activating form. A soluble form of BTF4 or B7-L1, or a receptor binding portion thereof, which is monomeric and thus cannot cross-link the receptor sufficiently to cause activation may be used to compete for receptor binding with naturally occurring BTF4 or B7-L1 and thus serve as an inhibitor of BTF4 or B7-L1 mediated signaling.

Biologically active portions of a BTF4 or B7-L1 protein include peptides comprising amino acid sequences sufficiently homologous to or derived from the amino acid sequence of the BTF4 or B7-L1 protein, which include less amino acids than the full length BTF4 or B7-L1 proteins, and exhibit at least one activity of a BTF4 or B7-L1 protein, preferably the ability to bind to a natural receptor. Typically, biologically active portions comprise a domain or motif with at least one activity of the BTF4 or B7-L1 protein. A biologically active portion of a BTF4 or B7-L1 protein can be a polypeptide which is, for example, at least about 100, 150, 200 or more amino acids in length.

Another type of agent useful for increasing BTF4 or B7-L1 signaling is an agonist of BTF4 or B7-L1. An agonist may, for instance, be a functional variant of the naturally occurring protein, a mimic or peptidomimetic, or a small molecule (e.g., less than 10 kDa). Preferably, the agonist exhibits the activity of BTF4 or B7-L1 required for the modulatory effect (e.g., binds the BTF4 or B7-L1 immunoinhibitory receptor in an analogous fashion as BTF4 or B7-L1 to activate signaling). For instance an agonist in soluble form may be multivalent. Alternatively an agonist may be linked to a solid or semi-solid substrate, or expressed on the surface of a cell, in sufficient number to promote receptor crosslinking and activation.

Variants of the BTF4 or B7-L1 proteins which serve as agonists can be generated by mutagenesis (e.g., amino acid substitution, amino acid insertion, or truncation of the BTF4 or B7-L1 protein). A variant of the BTF4 or B7-L1 protein which serves as an agonist must retain substantially the same, or a subset, of the biological activities of the naturally occurring form of a BTF4 or B7-L1 protein to inhibit immune cell activation. It may be advantageous to use a variant which retains only a subset of the biological activities of the naturally occurring form of BTF4 or B7-L1 protein in order to elicit specific biological effects by treatment with a variant of limited function. In one embodiment, treatment of a subject with a variant having a subset of the biological activities of the naturally occurring form of the protein has fewer side effects in a subject relative to treatment with the naturally occurring form of the BTF4 or B7-L1 protein. Variants that act as antagonists can also be made.

Variants of a BTF4 or B7-L1 protein can be identified by screening combinatorial libraries of mutants, such as truncation mutants, of a BTF4 or B7-L1 protein for the desired activity, (e.g., BTF4 or B7-L1 protein agonist or antagonist). In one embodiment, a variegated library of BTF4 or B7-L1 variants is generated by combinatorial mutagenesis at the nucleic acid level and is encoded by a variegated gene library. A variegated library of BTF4 or B7-L1 variants can be produced by, for example, enzymatically ligating a mixture of synthetic oligonucleotides into gene sequences such that a degenerate set of potential BTF4 or B7-L1 sequences is expressible as individual polypeptides, or alternatively, as a set of larger fusion proteins (e.g., for phage display) containing the set of BTF4 or B7-L1 sequences therein. There are a variety of methods which can be used to produce libraries of potential BTF4 or B7-L1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential BTF4 or B7-L1 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

B. Fusion Proteins

Another form of agonist or antagonist may be a fusion protein or chimeric protein derived from BTF4 or B7-L1, or a fragment of variant thereof. As used herein, a BTF4 or B7-L1 “chimeric protein” or “fusion protein” comprises a BTF4 or B7-L1 polypeptide, fragment, or functional variant thereof, operatively linked to a non-BTF4 or B7-L1 polypeptide. A “BTF4 or B7-L1 polypeptide” refers to a polypeptide having an amino acid sequence corresponding to BTF4 or B7-L1 polypeptide, whereas a “non-BTF4 or B7-L1 polypeptide” refers to a polypeptide having an amino acid sequence corresponding to a protein which is not substantially homologous to the BTF4 or B7-L1 protein, e.g., a protein which is different from the BTF4 or B7-L1 protein and which is derived from the same or a different organism. Within a BTF4 or B7-L1 fusion protein the BTF4 or B7-L1 polypeptide can correspond to all or a portion of a BTF4 or B7-L1 protein. In a preferred embodiment, a BTF4 or B7-L1 fusion protein comprises at least one biologically active portion of a BTF4 or B7-L1 protein, e.g., an extracellular domain of a BTF4 or B7-L1 protein. Within the fusion protein, the term “operatively linked” is intended to indicate that the BTF4 or B7-L1 polypeptide and the non-BTF4 or B7-L1 polypeptide are fused in-frame to each other. The non-BTF4 or B7-L1 polypeptide can be fused to the N-terminus or C-terminus of the BTF4 or B7-L1 polypeptide.

For example, in one embodiment, the fusion protein is a fusion protein in which BTF4 or B7-L1 amino acid sequences (e.g., the extracellular domain of a BTF4 or B7-L1 molecule) are fused to the Fc domain of an antibody molecule. In another embodiment, the fusion protein is a GST-B74 or GST-PD-1 fusion protein in which BTF4 or B7-L1 amino acid sequences are fused to the C-terminus of the GST sequences. In another embodiment, the fusion protein is a BTF4 or B7-L1-HA fusion protein in which the BTF4 or B7-L1 nucleotide sequence is inserted in a vector such as pCEP4-HA vector (Herrscher, R. F. et al. (1995) Genes Dev. 9:3067-3082) such that the BTF4 or B7-L1 sequences are fused in frame to an influenza hemagglutinin epitope tag. Such fusion proteins can facilitate the purification of a recombinant BTF4 or B7-L1 protein.

A BTF4 or B7-L1 fusion protein can be produced by recombinant expression of a nucleotide sequence encoding a first peptide having BTF4 or B7-L1 activity and a nucleotide sequence encoding second peptide corresponding to a moiety that alters the solubility, affinity, stability or valency of the first peptide, for example, an immunoglobulin constant region. Preferably, the first peptide consists of a portion of the BTF4 or B7-L1 polypeptide (e.g., a portion after cleavage of the signal sequence) of the sequence shown in SEQ ID NO: 2 or 4 that is sufficient to modulate an immune response. The second peptide can include an immunoglobulin constant region, for example, a human Cγ1 domain or Cγ4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). A resulting fusion protein may have altered BTF4 or B7-L1 solubility, binding affinity, stability and/or valency (i.e., the number of binding sites available per molecule) and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Particularly preferred BTF4 or B7-L1 Ig fusion proteins include the extracellular domain portion or variable region-like domain of a human BTF4 or B7-L1 coupled to an immunoglobulin constant region (e.g., the Fc region). The amino acid sequence of exemplary embodiments of a BTF4-Fc and a B7-L1-Fc fusion protein are shown in SEQ ID NO: 5 and 6, respectively. Such fusion proteins are bivalent and therefore have the potential to crosslink BTF4 or B7-L1 receptors in soluble form. Other embodiments of the fusion proteins may be monovalent, and thus lack the ability to crosslink BTF4 or B7-L1 in soluble form, however the linkage of a monovalent fusion protein to form a multi-valent entity, e.g., via crosslinking to a solid or semi-solid matrix or expression on the surface of a cell, will also result in an activating form of the fusion protein of BTF4 or B7-L1 respectively. The immunoglobulin constant region may contain genetic modifications which reduce or eliminate effector activity inherent in the immunoglobulin structure. For example, DNA encoding the extracellular portion of a BTF4 or B7-L1 polypeptide can be joined to DNA encoding the hinge, CH2 and CH3 regions of human IgGγ1 and/or IgGγ4 modified by site directed mutagenesis, e.g., as taught in WO 97/28267.

Preferably, a BTF4 or B7-L1 fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A BTF4 or B7-L1 encoding nucleic acid can be cloned into such an expression vector such that the fusion moiety is linked in-frame to the BTF4 or B7-L1 protein.

In another embodiment, the fusion protein is a BTF4 or B7-L1 protein containing a heterologous signal sequence at its N-terminus. In certain host cells (e.g., mammalian host cells), expression and/or secretion of BTF4 or B7-L1 can be increased through use of a heterologous signal sequence.

The BTF4 or B7-L1 fusion proteins of the invention can be incorporated into pharmaceutical compositions and administered to a subject in vivo. Use of activating forms of BTF4 or B7-L1 fusion proteins is useful therapeutically for the treatment of immunological disorders, e.g., autoimmune diseases, or in the case of inhibiting rejection of transplants. Soluble or crosslinked forms of the BTF4 or B7-L1-fusion proteins of the invention can be used as immunogens to produce anti-BTF4 or B7-L1 antibodies in a subject, to purify BTF4 or B7-L1 and in screening assays to identify molecules which inhibit the interaction of BTF4 or B7-L1 with its receptor.

Preferably, a BTF4 or B7-L1 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A BTF4 or B7-L1-encoding nucleic acid can be cloned into such an expression vector such that the fusion moiety is linked in-frame to the BTF4 or B7-L1 protein.

C. Antibodies

An antibody which specifically binds BTF4 or B7-L1 to prevent receptor binding also serves as an antagonist of BTF4 or B7-L1 mediated signaling. Production of such an antibody is within the ability of one of average skill in the art. As used herein, the term “antibody” includes immunoglobulin molecules and immunologically active portions of immunoglobulin molecules, for instance, molecules that contain an antigen binding site which binds (immunoreacts with) an antigen, such as Fab and F(ab′)₂ fragments, single chain antibodies, intracellular antibodies, scFv, Fd, or other fragments. Preferably, the antibody binds specifically or substantially specifically to the BTF4 or B7-L1 molecule. The antibody may be monoclonal or polyclonal. The terms “monoclonal antibodies” as used herein, refers to a population of antibody molecules that contain only one species of an antigen binding site capable of immunoreacting with a particular epitope of an antigen, whereas the term “polyclonal antibodies” refers to a population of antibody molecules that contain multiple species of antigen binding sites capable of interacting with a particular antigen.

Antibodies which bind to the BTF4 or B7-L1 receptor to crosslink and thus activate the receptor also serve as agents which increase BTF4 or B7-L:1 mediated signaling. Antibodies which bind to the BTF4 or B7-L1 receptor to prevent BTF4 or B7-L1 binding, but do not activate the receptor can be used as agents which inhibit BTF4 or B7-L1 mediated signaling. Production of such an antibody is within the ability to one of average skill in the art. Activation of a BTF4 or B7-L1 receptor can be assayed for instance by inhibition of co-stimulation in a T cell activation assay such as the type described in the Examples section below.

The use of recombinant anti-BTF4, anti-B7-L1, anti-BTF4-receptor, and anti-B7-L1-receptor antibodies, such as chimeric and humanized monoclonal antibodies, comprising both human and non-human portions, which can be made using standard recombinant DNA techniques, are also within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies which bind BTF4 or B7-L1, or their respective receptors, can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable genetic display package and vectors containing nucleic acid molecules encoding a second polypeptide chain of a single binding pair member using techniques known in the art (e.g., as described in U.S. Pat. No. 5,565,332, 5,871,907, or 5,733,743). The use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

Fully human antibodies can also be made using techniques that are known in the art. For example, Transgenic mice can be made using standard methods, e.g., according to Hogan, et al., “Manipulating the Mouse Embryo: A Laboratory Manual”, Cold Spring Harbor Laboratory, which is incorporated herein by reference, or are purchased commercially. Embryonic stem cells are manipulated according to published procedures (Teratocarcinomas and embryonic stem cells: a practical approach, Robertson, E. J. ed., IRL Press, Washington, D.C., 1987; Zjilstra et al. (1989) Nature 342:435438; and Schwartzberg et al. (1989) Science 246:799-803, each of which is incorporated herein by reference). Transgenic mice can be immunized using a purified or recombinant BTF4 or B7-L1 or a fusion protein comprising at least an immunogenic portion of the extracellular domain of BTF4 or B7-L1. Antibody reactivity can be measured using standard methods.

As an alternative to preparing monoclonal antibody-secreting hybridomas, anti BTF4 or B7-L1 antibodies (single chain Fv-like portions of antibodies) can be identified and isolated by screening a combinatorial library of human immunoglobulin sequences displayed on M13 bacteriophage (Winter et al. 1994 Annu. Rev. Immunol. 1994 12:433; Hoogenboom et al., 1998, Immunotechnology 4: 1). BTF4Fc or B7-L1Fc can be used to thereby isolate immunoglobulin library members that bind a BTF4 or B7-L1 polypeptide. Kits for generating and screening phage display libraries are commercially available and standard methods may be employed to generate the scFv (Helfrich et al. J. Immunol Methods 2000. 237: 131-45; Cardoso et al. Scand J. Immunol 2000. 51: 337-44).

In another embodiment, Ribosomal display can be used to replace bacteriophage as the display platform (see, e.g., Hanes et al. 2000. Nat. Biotechnol. 18:1287; Wilson et al. 2001. Proc. Natl. Acad. Sci. USA 98:3750; or Irving et al. 2001 J. Immunol. Methods 248:31. In yet another embodiment, cell surface libraries can be screened for antibodies (Boder et al. 2000. Proc. Natl. Acad. Sci. USA 97:10701; Daugherty et al. 2000 J. Immunol. Methods 243:211. Such procedures provide alternatives to traditional hybridoma techniques for the isolation and subsequent cloning of monoclonal antibodies.

D. Additional Modulators of BTF4 or B7-L1

Additional modulators of BTF4 or B7-L1 can also be identified. For example, libraries of fragments of a BTF4 or B7-L1 protein coding sequence can be used to generate a variegated population of BTF4 or B7-L1 fragments for screening and subsequent selection of variants of a BTF4 or B7-L1 protein. In one embodiment, a library of coding sequence fragments can be generated by treating a double stranded PCR fragment of a BTF4 or B7-L1 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the BTF4 or B7-L1 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of BTF4 or B7-L1 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify BTF4 or B7-L1 variants (Arkin and Youvan (1992) Proc. Natl. Acad. Sci. USA 89:7811-7815; Delagrave et al. (1993) Protein Eng. 6(3):327-331).

In one embodiment, cell based assays can be exploited to analyze a variegated BTF4 or B7-L1 library. For example, a library of expression vectors can be transfected into a cell line which ordinarily synthesizes and secretes BTF4 or B7-L1. The transfected cells are then cultured such that BTF4 or B7-L1 and a particular mutant BTF4 or B7-L1 are secreted and the effect of expression of the mutant on BTF4 or B7-L1 activity in cell supernatants can be detected, e.g., by any of a number of functional assays. Plasmid DNA can then be recovered from the cells which score for inhibition, or alternatively, potentiation of BTF4 or B7-L1 activity, and the individual clones further characterized.

Another form of agonist is a peptide analog or peptide mimetic of the BTF4 or B7-L1 protein. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p. 392; and Evans et al. (1987) J. Med. Chem. 30:1229, which are incorporated herein by reference) and are usually developed with the aid of computerized molecular modeling. Peptide mimetics that are structurally similar to BTF4 or B7-L1 or functional variants thereof, can be used to produce an equivalent effect. Generally, peptidomimetics are structurally similar to the paradigm polypeptide (BTF4 or B7-L1) but have one or more peptide linkages optionally replaced by a linkage selected from the group consisting of: —CH2NH—, —CH2S—, —CH2—CH2-, —CH═CH— (cis and trans), —COCH2-, —CH(OH)CH2-, and —CH2SO—. This is accomplished by the skilled practitioner by methods known in the art which are further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2—S); Hann, M. M. (1982) J. Chem. Soc. Perkin Trans. L 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2—); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptides, including, for example: more economical production, greater chemical stability, enhanced pharmacological properties (half-life, absorption, potency, efficacy, etc.), altered specificity (e.g., a broad-spectrum of biological activities), and reduced antigenicity.

Systematic substitution of one or more amino acids of either BTF4 or B7-L1 amino acid sequence, or a functional variant thereof, with a D-amino acid of the same type (e.g., D-lysine in place of L-lysine) can be used to generate a peptide agonist which has increased stability. In addition, constrained peptides comprising a BTF4 or B7-L1 amino acid sequence, a functional variant thereof, or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide, to produce an agonist.

A variant of BTF4 or B7-L1 which retains only partial function may serve as an antagonist. An antagonist of a BTF4 or B7-L1 protein can inhibit one or more of the activities of the naturally occurring form of the BTF4 or B7-L1 protein by, for example, competitively modulating a cellular activity of a BTF4 or B7-L1 protein. Such a variant is produced by similar methods as variants discussed above, and is identified functionally. A variant which is an antagonist of either BTF4 or B7-L1 retains the ability to bind the immunoinhibitory receptor, but lacks the ability to induce signal transduction of the inhibitory signal. Another form of antagonist is a peptidomimetic of BTF4 or B7-L1 which competes for binding of the respective receptor, but does not active the receptor to induce a response. A small molecule which binds BTF4 or B7-L1 to prevent its activity, (e.g., binding to the immunoinhibitory receptor), or which binds to the corresponding immunoinhibitory receptor to prevent signaling, also serves an antagonist.

Peptides can be produced, typically by direct chemical synthesis, and used as agonists or antagonists of an interaction of BTF4 or B7-L1 with the corresponding immunoinhibitory receptor. Peptides may be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, and desirable pharmacokinetic properties.

III. Recombinant Expression Vectors and Host Cells

Nucleic acid molecules encoding a modulator of BTF4 or B7-L1 mediated signaling can be contained in vectors, preferably expression vectors. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Recombinant expression vectors can comprise a nucleic acid molecule of the invention in a form suitable for expression, e.g., constitutive or inducible expression, of a PD-1 or B74 molecule in the indicator cell(s) of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cell and those which direct expression of the nucleotide sequence only in certain host cells (e.g., tissue-specific regulatory sequences). It should be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., BTF4 or B7-L1 family proteins, mutant forms of BTF4 or B7-L1 proteins, fusion proteins, and the like).

The recombinant expression vectors of the invention can be designed for expression of the modulatory agent in prokaryotic or eukaryotic cells. For example, the modulatory agents can be expressed in bacterial cells such as E. coli, insect cells (using baculovirus expression vectors) yeast cells or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified proteins can be utilized in BTF4 or B7-L1 activity assays, (e.g., direct assays or competitive assays described in detail below), or to generate antibodies specific for BTF4 or B7-L1 proteins, for example.

Examples of suitable inducible non-fusion E. coli expression vectors include pTrc (Amann et al., (1988) Gene 69:301-315) and pET 11d (Studier et al. (1990) Methods Enzymol. 185:60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S. (1990) Methods Enzymol. 185:119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the modulatory agent expression vector is a yeast expression vector. Examples of vectors for expression in yeast S. cerevisiae include pYepSec1 (Baldari, et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and picZ (Invitrogen Corp, San Diego, Calif.).

Alternatively, a modulatory agent can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow, V. A. and Summers, M. D. (1989) Virology 170:31-39).

In yet another embodiment, a nucleic acid encoding a modulatory agent is expressed in mammalian cells using a mammalian expression vector. Examples of mammalian expression vectors include pMex-NeoI, pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al. Molecular Cloning: A Laboratory Manual. 2nd, ed., Cold Spring Harbor Laboratory, Cold Spring Harbor Laboratory Press, Cold Spring Harbor, N.Y., 1989.

Some mammalian expression vectors are capable of directing expression of the inserted nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, ed. Nouer, L., CRC, Boca Raton, Fla., pp 167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel and Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Accordingly, in another embodiment, the invention provides a recombinant expression vector in which a BTF4 or B7-L1 DNA is operatively linked to an inducible eukaryotic promoter, thereby allowing for inducible expression of a modulatory agent in eukaryotic cells.

A DNA molecule may also be cloned into the expression vector in an antisense orientation. That is, the DNA molecule is operatively linked to a regulatory sequence in a manner which allows for expression (by transcription of the DNA molecule) of an RNA molecule which is antisense to a natural mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al. (1986) “Antisense RNA as a molecular tool for genetic analysis” Reviews—Trends in Genetics, Vol. 1(1).

The invention further pertains to host cells into which a recombinant expression vector encoding a modulator of BTF4 or B7-L1 signaling has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a modulatory agent can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a modulator of BTF4 or B7-L1 mediated signaling or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a modulatory agent. Accordingly, the invention further provides methods for producing a modulatory agent using the host cells of the invention. In one embodiment, the method comprises culturing the host cell of invention (into which a recombinant expression vector encoding a modulatory agent has been introduced) in a suitable medium such that the modulatory agent protein is produced. In another embodiment, the method further comprises isolating a modulatory agent from the medium or the host cell.

Certain host cells can also be used to produce non-human transgenic animals. For example, in one embodiment, a host cell is a fertilized oocyte or an embryonic stem cell into which modulatory agent coding sequences have been introduced. Such host cells can then be used to create non-human transgenic animals in which exogenous modulatory agent sequences have been introduced into their genome or homologous recombinant animals in which endogenous BTF4 or B7-L1 sequences have been altered to produce a the desired modulatory effect. Such animals are useful for studying the function and/or activity of a BTF4 or B7-L1 polypeptide and for identifying and/or evaluating modulators of BTF4 or B7-L1 mediated signaling. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous modulatory agent gene has been altered by homologous recombination between the endogenous gene and an exogenous DNA molecule introduced into a cell of the animal, e.g., an embryonic cell of the animal, prior to development of the animal.

A transgenic animal can be created by introducing a modulatory agent-encoding nucleic acid molecule into the male pronucleus of a fertilized oocyte, e.g., by microinjection, retroviral infection, and allowing the oocyte to develop in a pseudopregnant female foster animal. The modulatory agent DNA sequence described above can be introduced as a transgene into the genome of a non-human animal. When a BTF4 or B7-L1 molecule, or a variant thereof is used as a modulatory agent, that molecule may be derived from a nonhuman homologue of a human BTF4 or B7-L1 gene, such as a mouse or rat BTF4 or B7-L1 gene, and the derived gene product can be used as a transgene. Alternatively, a BTF4 or B7-L1 gene homologue, such as another BTF4 or B7-L1 family member, can be isolated based on hybridization to the BTF4 or B7-L1 family cDNA sequences of SEQ ID NO: 1 or 3 (described further in subsection I above) and used to derived the modulatory agent. Intronic sequences and polyadenylation signals can also be included in the transgene to increase the efficiency of expression of the transgene. A tissue-specific regulatory sequence(s) can be operably linked to a modulatory agent transgene to direct expression of a modulatory agent protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B. Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the modulatory agent transgene in its genome and/or expression of the modulatory agent mRNA in tissues or cells of the animals. A transgenic founder animal can then be used to breed additional animals carrying the transgene. Moreover, transgenic animals carrying a transgene encoding a modulatory agent can further be bred to other transgenic animals carrying other transgenes.

To create a homologous recombinant animal, a vector is prepared which contains at least a portion of a modulatory agent gene into which a deletion, addition or substitution has been introduced to thereby alter, e.g., functionally disrupt, the BTF4 or B7-L1 gene. The BTF4 B7-L1 gene can be a human gene, but more preferably, is a non-human homologue of a human BTF4 or B7-L1 gene (e.g., a cDNA isolated by stringent hybridization with the nucleotide sequence of SEQ ID NO: 1 or 3). For example, a mouse BTF4 or B7-L1 gene can be used to construct a homologous recombination vector suitable for altering an endogenous BTF4 or B7-L1 gene in the mouse genome. In a preferred embodiment, the vector is designed such that, upon homologous recombination, an endogenous modulatory agent gene (e.g., BTF4 or B7-L1) is functionally disrupted (i.e., no longer encodes a functional protein; also referred to as a “knock out” vector). Alternatively, the vector can be designed such that, upon homologous recombination, the endogenous modulatory agent gene is mutated or otherwise altered but still encodes a functional protein (e.g., the upstream regulatory region can be altered to thereby alter the expression of the endogenous modulatory agent protein). In the homologous recombination vector, the altered portion of the modulatory agent gene is flanked at its 5′ and 3′ ends by additional nucleic acid sequence of the modulatory agent gene to allow for homologous recombination to occur between the exogenous modulatory agent gene carried by the vector and an endogenous modulatory agent gene in an embryonic stem cell. The additional flanking modulatory agent nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced modulatory agent gene has homologously recombined with the endogenous modulatory agent gene are selected (see, e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, Robertson, E. J., ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Curr. Opin. Biotechnol. 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In addition to the foregoing, the ordinarily skilled artisan will appreciate that other approaches known in the art for homologous recombination can be applied to the instant invention. Enzyme-assisted site-specific integration systems are known in the art and can be applied to integrate a DNA molecule at a predetermined location in a second target DNA molecule. Examples of such enzyme-assisted integration systems include the Cre recombinase-lox target system (e.g., as described in Baubonis, W. and Sauer, B. (1993) Nucl. Acids Res. 21:2025-2029; and Fukushige, S. and Sauer, B. (1992) Proc. Natl. Acad. Sci. USA 89:7905-7909) and the FLP recombinase-FRT target system (e.g., as described in Dang, D. T. and Perrimon, N. (1992) Dev. Genet. 13:367-375; and Fiering, S. et al. (1993) Proc. Natl. Acad. Sci. USA 90:8469-8473). Tetracycline-regulated inducible homologous recombination systems, such as described in PCT Publication No. WO 94/29442 and PCT Publication No. WO 96/01313, also can be used.

For example, in another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Uses and Methods of the Invention

The BTF4 and/or B7-L1 modulatory agents, e.g., the nucleic acid molecules, proteins, protein homologues, and antibodies described herein, can be used in one or more of the following methods: a) screening assays; b) detection assays; c) predictive medicine (e.g., diagnostic assays, prognostic assays, monitoring clinical trials, and pharmacogenetics) as well as in methods of modulating immune cell activation. The isolated nucleic acid molecules of the invention can be used, for example, to express BTF4 or B7-L1 protein, or a modified version thereof (e.g., mutated, or truncated, or expressed as a fusion protein), for instance via a recombinant expression vector in a host cell in gene therapy applications, to detect BTF4 or B7-L1 mRNA (e.g., in a biological sample) or a genetic alteration in a BTF4 or B7-L1 gene, and to modulate BTF4 or B7-L1 mediated signaling, as described further below. The BTF4 or B7-L1 proteins can be used to treat disorders which involve aberrant BTF4 or B7-L1 mediated signaling (e.g., caused by insufficient or excessive production of BTF4 or B7-L1 inhibitors). In addition, the BTF4 or B7-L1 proteins can be used to screen for naturally occurring BTF4 or B7-L1 regulators, to screen for drugs or compounds which modulate BTF4 or B7-L1 mediated signaling, as well as to treat disorders characterized by insufficient or excessive production of BTF4 or B7-L1 protein or production of BTF4 or B7-L1 protein forms which have decreased or aberrant activity compared to BTF4 or B7-L1 wild type protein, both of which would result in abnormally low amounts of BTF4 or B7-L1 mediated signaling. Moreover, the anti-BTF4 or B7-L1 antibodies of the invention can be used to detect and isolate BTF4 or B7-L1 proteins (e.g., from an individual for diagnosis purposes), regulate the bioavailability of BTF4 or B7-L1 proteins, and modulate BTF4 or B7-L1 activity (e.g., by modulating the interaction of BTF4 or B7-L1 with their respective receptor).

A. Screening Assays

Another aspect of the invention relates to a method (also referred to herein as a “screening assay”) for identifying an agent (e.g., peptides, peptidomimetics, small molecules or other drugs) that modulates BTF4 or B7-L1 signaling, that is to say agents which have a stimulatory or inhibitory effect on BTF4 or B7-L1 mediated signaling. The agent is identified from one or more test agents (also referred to herein as candidate or test compounds) which are assayed for the ability to modulate BTF4 or B7-L1 signaling with a standard in vitro assay for immune response wherein the immune response is downregulated by the presence of BTF4 or B7-L1. A number of suitable readouts of immune cell activation (e.g., cell proliferation or effector function such as antibody production, cytokine production, and phagocytosis) in the presence of BTF4 or B7-L1 exist in the art. One commonly used assay is a T cell activation assay.

For example, T cells are manipulated by standard methods to produce activated T cells in the presence of BTF4 or B7-L1 to downmodulate T cell activation. A comparative change in the BTF4 or B7-L1 mediated downregulation of the immune response in the presence of the test agent indicates the test agent is a modulator of the BTF4 or B7-L1 signaling. Inhibition of BTF4 or B7-L1 mediated downregulation of the immune response results in a statistically significant and reproducible increase in the immune response as measured by the assay. Similarly, augmentation of BTF4 or B7-L1 mediated downregulation can also be measured. Agents that block or inhibit interaction of BTF4 or B7-L1 with their corresponding immunoinhibitory receptor, as well as agents that promote a BTF4 or B7-L1-mediated inhibitory signal can be identified by their ability to modulate immune cell activation, e.g., proliferation and/or effector function or to induce anergy when added to an in vitro assay.

In one embodiment, the invention provides assays for screening candidate or test compounds which bind to or modulate the expression and/or activity of a BTF4 or B7-L1 protein or polypeptide or biologically active portion thereof, e.g., modulate the ability of BTF4 or B7-L1 polypeptide to interact with its receptor or an interactor molecule (e.g., an intracellular interactor molecule) to affect regulation of BTF4 or B7-L1 mediated signaling. The test compounds of the present invention can be, for instance, any of the compounds described above. Such compounds may be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay. In one embodiment, such an assay comprises contacting a cell bearing BTF4 or B7-L1 or a cell expressing a BTF4 or B7-L1 receptor with a test compound and determining the ability of the test compound to modulate (e.g., upregulate or downregulate BTF4 or B7-L1 mediated signaling. Determining the ability of the test compound to modulate BTF4 or B7-L1 mediated signaling can be accomplished, for example, by determining the ability of the BTF4 or B7-L1 protein to bind to or interact with their respective receptors. Determining the ability of the BTF4 or B7-L1 protein to bind to or interact with its binding partner can be accomplished, for instance by measuring direct binding. Alternatively, induction of BTF4 or B7-L1 signaling can be measured. This can be accomplished using methods known in the art. For example, the activation state of the cell can be examined by measuring, e.g., cell surface marker expression, second messenger generation, or parameters of cellular activation (e.g., using a costimulation in a T cell activation assay such as described in the Examples section below).

In a direct binding assay, the BTF4 or B7-L1 protein, or a modified version or mimetic thereof (or their respective receptors) can be coupled with a radioisotope or enzymatic label such that binding of the BTF4 or B7-L1 protein to a BTF4 or B7-L1 target molecule can be determined by detecting the labeled protein in a complex. For example, BTF4 or B7-L1 molecules, e.g., BTF4 or B7-L1 proteins, can be labeled with ¹²⁵I, ³⁵S, ¹⁴C, or ³H, either directly or indirectly, and the radioisotope detected by direct counting of radioemmission or by scintillation counting. Alternatively, BTF4 or B7-L1 molecules can be enzymatically labeled with, for example, horseradish peroxidase, alkaline phosphatase, or luciferase, and the enzymatic label detected by determination of conversion of an appropriate substrate to product.

A test agent or compound may also function to inhibit BTF4 or B7-L1 mediated signaling by inhibiting the interaction between BTF4 or B7-L1 and its receptor. Such an activity of a test agent or compound to modulate the interaction between BTF4 or B7-L1 and its receptor can be determined without the labeling of any of the interactants. For example, a microphysiometer can be used to detect the interaction of BTF4 or B7-L1 with its receptor without the labeling of either BTF4 or B7-L1 or the receptor (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In a preferred embodiment, determining the ability of a test agent to induce or inhibit BTF4 or B7-L1 mediated signaling can be accomplished by measuring the effect of the test agent on the ability of BTF4 or B7-L1 to mediate signaling in an immune cell. BTF4 or B7-L1 mediated signaling can be determined, for instance, by detecting induction of a cellular second messenger (e.g., tyrosine kinase activity), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the BTF4 or B7-L1 receptor.

In another embodiment, the assay is a cell-free assay. For example, a BTF4 or B7-L1 protein or biologically active portion thereof can be contacted with a test agent and the ability of the test agent to modulate (e.g., stimulate or inhibit) the ability of the BTF4 or B7-L1 protein or biologically active portion thereof to signal via the appropriate receptor is determined. Determining the ability of the test agent to modulate the activity of BTF4 or B7-L1 can be accomplished, for example, by determining its effect on the ability of BTF4 or B7-L1 to bind to its receptor. Determining the ability of the BTF4 or B7-L1 protein to bind its receptor can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, a cell-free assay involves contacting a BTF4 or B7-L1 protein or biologically active portion thereof with a known compound which binds the BTF4 or B7-L1 protein to form an assay mixture, contacting the assay mixture with a test compound, determining the ability of the test compound to interact with the BTF4 or B7-L1 protein. The ability of the test compound to interact with the BTF4 or B7-L1 protein can be determined by measuring the ability of the BTF4 or B7-L1 protein to preferentially bind to or modulate the activity of a BTF4 or B7-L1 target molecule, e.g., a receptor.

In an alternative embodiment, determining the ability of the test compound to modulate the activity of a BTF4 or B7-L1 protein can be accomplished by determining the ability of the test compound to modulate the activity of a molecule that functions downstream of the BTF4 or B7-L1 receptor, e.g., by interacting with the cytoplasmic domain of the receptor. For example, levels of second messengers can be determined, the activity of the interactor molecule on an appropriate target can be determined, or the binding of the interactor to an appropriate target can be determined as previously described.

In another embodiment, modulators of BTF4 or B7-L1 expression (e.g., in an antigen presenting cell) are identified in a method wherein a cell is contacted with a test agent and the expression of BTF4 or B7-L1 mRNA or protein in the cell is determined. The level of expression of BTF4 or B7-L1 mRNA or protein in the presence of the test agent can be compared to the level of expression of BTF4 or B7-L1 mRNA or protein in the absence of the test agent. The test agent can then be identified as a modulator of BTF4 or B7-L1 expression based on this comparison. For example, when expression of BTF4 or B7-L1 mRNA or protein is greater (e.g., statistically significantly greater) in the presence of the test agent than in its absence, the test agent is identified as a stimulator of BTF4 or B7-L1 mRNA or protein expression. Alternatively, when expression of BTF4 or B7-L1 mRNA or protein is less (e.g., statistically significantly less) in the presence of the test agent than in its absence, the test agent is identified as an inhibitor of BTF4 or B7-L1 mRNA or protein expression. The level of BTF4 or B7-L1 mRNA or protein expression in the cells can be determined by methods described herein for detecting BTF4 or B7-L1 mRNA or protein.

In yet another aspect of the invention, the BTF4 or B7-L1 proteins, preferably in membrane bound form, can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins (“BTF4 or B7-L-1 binding proteins” or “BTF4 or B7-L1 bp”), which bind to or interact with BTF4 or B7-L1 and are involved in BTF4 or B7-L1 activity. Such BTF4 or B7-L1 binding proteins are also likely to be involved in the modulation of signals by the BTF4 or B7-L1 proteins or BTF4 or B7-L1 targets as, for example, upstream or downstream elements of a BTF4 or B7-L1 mediated signaling pathway.

The two-hybrid system is based on the modular nature of most transcription factors, which consist of separable DNA-binding and activation domains. Briefly, the assay utilizes two different DNA constructs. In one construct, the gene that codes for a BTF4 or B7-L1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the BTF4 or B7-L1 protein.

This invention further pertains to novel agents identified by the above-described screening assays. Furthermore, this invention pertains to uses of novel agents identified by the above-described screening assays or methods as described herein.

B. Detection Assays

Portions or fragments of the modulators of BTF4 or B7-L1, (e.g., protein or nucleic acid sequences) identified by the methods described herein can be used in numerous ways as reagents. For example, nucleic acid sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of nucleotide sequences functioning as or encoding a modulatory agent, described herein, can be used to map the location of the corresponding genes on a chromosome. The mapping of such sequences to chromosomes is an important first step in correlating such sequences with genes associated with disease.

Briefly, genes can be mapped to chromosomes by preparing PCR primers (preferably 15-25 bp in length) from nucleotide sequences. Computer analysis of sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio, P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using nucleotide sequences to design oligonucleotide primers, sublocalization can be achieved with panels of fragments from specific chromosomes. Other mapping strategies which can similarly be used to map a sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in McKusick, V., Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature 325:783-787.

Moreover, differences in the DNA sequences between individuals affected and unaffected with a disease associated with the gene of interest can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

C. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining BTF4 or B7-L1 protein and/or nucleic acid expression as well as BTF4 or B7-L1 activity, in the context of a biological sample (e.g., blood, serum, cells, tissue) to thereby determine whether an individual is afflicted with a disease or disorder, or is at risk of developing a disorder, associated with aberrant BTF4 or B7-L1 expression or activity which leads to aberrant BTF4 or B7-L1 mediated signaling. The invention also provides for prognostic (or predictive) assays for determining whether an individual is at risk of developing a disorder associated with BTF4 or B7-L1 protein, nucleic acid expression or activity. For example, mutations in a BTF4 or B7-L1 gene can be assayed in a biological sample for activity compared to wild type BTF4 or B7-L1. Such assays can be used for prognostic or predictive purpose to thereby prophylactically treat an individual prior to the onset of a disorder characterized by or associated with aberrant BTF4 or B7-L1 signaling.

Another aspect of the invention pertains to monitoring the influence of agents (e.g., drugs, compounds) on the expression or activity of BTF4 or B7-L1 in clinical trials.

These and other agents are described in further detail in the following sections.

D. Diagnostic Assays

An exemplary method for detecting the presence or absence of BTF4 or B7-L1 protein or nucleic acid in a biological sample involves obtaining a biological sample from a test subject and contacting the biological sample with a compound or an agent capable of detecting BTF4 or B7-L1 protein or nucleic acid (e.g., mRNA, genomic DNA) that encodes BTF4 or B7-L1 protein such that the presence of BTF4 or B7-L1 protein or nucleic acid is detected in the biological sample. A preferred agent for detecting BTF4 or B7-L1 mRNA or genomic DNA is a labeled nucleic acid probe capable of hybridizing to BTF4 or B7-L1 mRNA or genomic DNA. The nucleic acid probe can be, for example, a human BTF4 or B7-L1 nucleic acid molecule, such as the nucleic acid molecule of SEQ ID NO: 1 or 3 or a portion thereof, such as an oligonucleotide of at least 15, 30, 50, 100, 250 or 500 nucleotides in length and sufficient to specifically hybridize under stringent conditions to BTF4 or B7-L1 mRNA or genomic DNA. Other suitable probes for use in the diagnostic assays of the invention are described herein.

A preferred agent for detecting BTF4 or B7-L1 protein is an antibody capable of binding to BTF4 or B7-L1 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect BTF4 or B7-L1 mRNA, protein, or genomic DNA in a biological sample in vitro as well as in vivo. For example, in vitro techniques for detection of BTF4 or B7-L1 mRNA include Northern hybridizations and in situ hybridizations. In vitro techniques for detection of BTF4 or B7-L1 protein include enzyme linked immunosorbent assays (ELISAs), Western blots, immunoprecipitations and immunofluorescence. In vitro techniques for detection of BTF4 or B7-L1 genomic DNA include Southern hybridizations. Furthermore, in vivo techniques for detection of BTF4 or B7-L1 protein include introducing into a subject a labeled anti-BTF4 or B7-L1 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting BTF4 or B7-L1 protein, mRNA, or genomic DNA, such that the presence of BTF4 or B7-L1 protein, mRNA or genomic DNA is detected in the biological sample, and comparing the presence of BTF4 or B7-L1 protein, mRNA or genomic DNA in the control sample with the presence of BTF4 or B7-L1 protein, mRNA or genomic DNA in the test sample.

The invention also encompasses kits for detecting the presence of BTF4 or B7-L1 in a biological sample. For example, the kit can comprise a labeled compound or agent capable of detecting BTF4 or B7-L1 protein or mRNA in a biological sample; means for determining the amount of BTF4 or B7-L1 in the sample; and means for comparing the amount of BTF4 or B7-L1 in the sample with a standard. The compound or agent can be packaged in a suitable container. The kit can further comprise instructions for using the kit to detect BTF4 or B7-L1 protein or nucleic acid.

Aberrant BTF4 or B7-L1 signaling may also result from a condition which does not involve aberrant expression of BTF4 or B7-L1. In such a situation, BTF4 or B7-L1 signaling may be studied to identify the presence of a disorder. Thus, the present invention also relates to the detection of BTF4 or B7-L1 mediated signaling in a subject and quantitation of said signaling with respect to a normal range which is to be expected in a healthy individual. Detection of BTF4 or B7-L1 mediated signaling is performed for instance, in vitro, with immune cells isolated from a test subject, by procedures for the detection and quantitation of BTF4 or B7-L1 mediated signaling, such as those detailed in the Examples section below. Such detection is performed using cells of the test subject, and compared to the results obtained from identical detection(s) performed using cells of one or more healthy individuals, to establish a normal range.

E. Prognostic Assays

The diagnostic methods described herein can furthermore be utilized to identify subjects having or at risk of developing a disease or disorder associated with aberrant BTF4 or B7-L1 mediated signaling (e.g., resulting from aberrant BTF4 or B7-L1 expression or activity). For example, the assays described herein, such as the preceding diagnostic assays or the following assays, can be utilized to identify a subject having or at risk of developing a disorder associated with aberrant BTF4 or B7-L1 mediated signaling. One such method for identifying a disease or disorder associated with aberrant BTF4 or B7-L1 expression or activity involves a test sample being obtained from a subject and the detection of BTF4 or B7-L1 protein or nucleic acid molecule (e.g., mRNA, genomic DNA) in the sample, wherein the presence of abnormally high or low levels of BTF4 or B7-L1 protein or nucleic acid, or BTF4 or B7-L1 activity, is diagnostic for a subject having or at risk of developing a disease or disorder associated with aberrant BTF4 or B7-L1 expression or activity. As used herein, a “test sample” refers to a biological sample obtained from a subject of interest. For example, a test sample can be a biological fluid (e.g., serum), cell sample, or tissue. BTF4 or B7-L1 activity is determined by the amount of BTF4 or B7-L1 mediated signaling which results from the activity of a BTF4 or B7-L1 molecule (e.g., a mutant molecule), and can be determined, for instance, by in vitro assays designed to measure BTF4 or B7-L1 mediated signaling described herein.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant BTF4 or B7-L1 mediated signaling. Thus, the present invention provides methods for determining whether a subject can be effectively treated with an agent for a disorder associated with aberrant BTF4 or B7-L1 expression or activity in which a test sample is obtained and BTF4 or B7-L1 protein or nucleic acid expression, activity, or induced expression is detected. In some instances the abundance of BTF4 or B7-L1 protein or nucleic acid expression or activity is directly related to the BTF4 or B7-L1 mediated signaling of a subject. In this and possibly in other instances, administration of an agent to treat the aberrant BTF4 or B7-L1 expression or activity will be sufficient to treat the aberrant BTF4 or B7-L1 mediated signaling. a disorder associated with aberrant BTF4 or B7-L1 expression or activity).

Genetic alterations in a subject's BTF4 or B7-L1 gene may be detected by methods known in the art, to thereby determine if the subject with the altered gene is at risk for a disorder associated with aberrant BTF4 or B7-L1 signaling. In preferred embodiments, the methods include detecting, in a sample of cells from the subject, the presence or absence of a genetic alteration characterized by at least one of an alteration affecting the integrity of a gene encoding a BTF4 or B7-L1 protein, or the mis-expression of the BTF4 or B7-L1 gene. For example, such genetic alterations can be detected by ascertaining the existence of at least one of 1) a deletion of one or more nucleotides from a BTF4 or B7-L1 gene; 2) an addition of one or more nucleotides to a BTF4 or B7-L1 gene; 3) a substitution of one or more nucleotides of a BTF4 or B7-L1 gene, 4) a chromosomal rearrangement of a BTF4 or B7-L1 gene; 5) an alteration in the level of a messenger RNA transcript of a BTF4 or B7-L1 gene, 6) aberrant modification of a BTF4 or B7-L1 gene, such as of the methylation pattern of the genomic DNA, 7) the presence of a non-wild type splicing pattern of a messenger RNA transcript of a BTF4 or B7-L1 gene, 8) a non-wild type level of a BTF4 or B7-L1 protein, 9) allelic loss of a BTF4 or B7-L1 gene, and 10) inappropriate post-translational modification of a BTF4 or B7-L1 protein. As described herein, there are a large number of assay techniques known in the art which can be used for detecting alterations in a BTF4 or B7-L1 gene. A preferred biological sample is a tissue or serum sample isolated by conventional means from a subject, e.g., a cardiac tissue sample.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the BTF4 or B7-L1 gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a BTF4 or B7-L1 gene under conditions such that hybridization and amplification of the BTF4 or B7-L1 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Biotechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a BTF4 or B7-L1 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in BTF4 or B7-L1 can be identified by hybridizing a sample and control nucleic acids, e.g., DNA or RNA, to high density arrays containing hundreds or thousands of oligonucleotides probes (Cronin, M. T. et al. (1996) Hum. Mutat. 7:244-255; Kozal, M. J. et al. (1996) Nat. Med. 2:753-759). For example, genetic mutations in BTF4 or B7-L1 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the BTF4 or B7-L1 gene and detect mutations by comparing the sequence of the sample BTF4 or B7-L1 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the BTF4 or B7-L1 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type BTF4 or B7-L1 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in BTF4 or B7-L1 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a B74 sequence, e.g., a wild-type BTF4 or B7-L1 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility can be used to identify mutations in BTF4 or B7-L1 genes. For example, single strand conformation polymorphism (SSCP) can be used to detect differences in electrophoretic mobility between mutant and wild type nucleic acids (Orita et al. (1989) Proc Natl. Acad. Sci. USA: 86:2766, see also Cotton (1993) Mutat. Res. 285:125-144; and Hayashi (1992) Genet. Anal. Tech. Appl. 9:73-79). Single-stranded DNA fragments of sample and control BTF4 or B7-L1 nucleic acids can be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA can be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner et al. (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a BTF4 or B7-L1 gene.

Furthermore, any cell type or tissue in which BTF4 or B7-L1 is expressed can be utilized in the prognostic assays described herein.

V. Methods of Treatment:

The present invention provides for both prophylactic and therapeutic methods of treating a subject at risk of (or susceptible to) a disorder or having a disorder associated with aberrant BTF4 or B7-L1 mediated signaling. One aspect of the present invention relates to a method for modulating an immune response with an agent that modulates BTF4 or B7-L1 mediated signaling. In one embodiment, the agent increases the BTF4 or B7-L1 signaling to downregulate the immune response. It may be advantageous to manipulate both B7-L1 and BTF4 signaling to produce the desired upregulation or downregulation of the immune response. In a preferred embodiment, anergy is induced in the immune cell.

Preferably, the modulatory agent is contacted with an immune cell, (e.g., a T cell). Contacting may occur in vivo or in vitro. Contact in vitro may be performed for instance as a matter of course in the preparation of a therapeutic (e.g., for an ex vivo therapeutic) or for modulation of cells ex vivo. Contacting in vivo may, for instance, be as a matter of course in the performance of treatment of an individual who would benefit from upregulation or down regulation of an immune response, discussed in greater detail below.

The therapeutic and prophylactic methods described herein are to be used on an individual who would benefit from the modulation of an immune response.

A. Prophylactic Methods

In one aspect, the invention provides a method for preventing in a subject, a disease or condition associated with an aberrant BTF4 or B7-L1 signaling, by prophylactically administering to the subject an agent which modulates BTF4 or B7-L1 mediated signaling. Subjects at risk for a disease which is caused or contributed to by aberrant BTF4 or B7-L1 signaling can be identified by, for example, any or a combination of diagnostic or prognostic assays as described herein. Administration of a prophylactic agent can occur prior to the manifestation of symptoms characteristic of BTF4 or B7-L1 signaling aberrancy, such that a disease or disorder is prevented or, alternatively, delayed in its progression. Depending on the type of BTF4 or B7-L1 signaling aberrancy or condition, for example, a BTF4 or B7-L1 polypeptide, BTF4 or B7-L1 agonist or BTF4 or B7-L1 antagonist can be used for treating the subject. The appropriate agent can be determined based on clinical indications and can be identified, e.g., using screening assays described herein.

B. Therapeutic Methods

BTF4 and B7-L1 has been demonstrated to inhibit the costimulation and proliferation of activated immune cells and to transmit an inhibitory signal to immune cells. Accordingly, this signaling can be modulated in order to modulate the immune response. It should be understood that inhibition of BTF4 or B7-L1 mediated signaling results in upregulation of immune responses, whereas stimulation or activation of BTF4 or B7-L1 mediated signaling results in downregulation of immune responses.

The present invention can be used to modulate both primary and secondary immune responses in the individual. Experiments detailed in the Exemplification section below indicate that modulation of BTF4 and B7-L1 signaling directly modulates the primary T cell immune response. Such modulation of T cell activity in an individual will further affect secondary immune responses. In addition to the direct modulation of the T cell immune response of an individual, modulation of the BTF4 or B7-L1 signaling is also expected to directly result in analogous modulation of immune response mounted by other immune cells which express the BTF4 or B7-L1 receptor, to directly affect the immune response of the individual. The immune response of the individual can be determined by assaying antibody production, immune cell proliferation, the release of cytokines, the expression of cell surface markers, cytotoxicity, etc.

Modulatory methods of the invention involve contacting a cell with a modulator of BTF4 or B7-L1 mediated signaling (e.g., an agent that modulates expression or activity of BTF4 or B7-L1, an agent that mimics or inhibits BTF4 or B7-L1, or an agent that modulates the interaction of BTF4 or B7-L1 with its receptor). Agents which modulate BTF4 or B7-L1 mediated signaling, also referred to herein as modulatory agents, are described in detail above. The nature and mechanism of the modulatory agent will dictate which cell is contacted with the modulatory agent to produce the desired effect on the immune response.

Modulatory agents can be administered in vitro (e.g., by contacting the cell with the agent) or, alternatively, in vivo (e.g., by administering the agent to a subject). As such, the present invention provides methods of treating an individual afflicted with a disease or disorder that would benefit from modulation of a an immune response, e.g., a disorder which would benefit from up or downmodulation of the immune response, or which is characterized by aberrant expression or activity of a BTF4 or B7-L1 protein or nucleic acid molecule. In one embodiment, the method involves administering an agent (e.g., an agent identified by a screening assay described herein), or combination of agents that modulates (e.g., upregulates or downregulates) BTF4 or B7-L1 mediated signaling.

Upregulation of BTF4 or B7-L1 mediated signaling is desirable in situations in which an immune response is abnormally high (e.g., in situations in which BTF4 or B7-L1 signaling is abnormally low) and/or in which an enhanced immune response via increased BTF4 or B7-L1 signaling is likely to have a beneficial effect. Likewise, inhibition of BTF4 or B7-L1 mediated signaling is desirable in situations in which an immune response is abnormally weak and/or in which decreased BTF4 or B7-L1 signaling is likely to have a beneficial effect.

Exemplary agents for use in upmodulating an immune response by decreasing BTF4 or B7-L1 mediated signaling (BTF4 or B7-L1 antagonists) include, without limitation: antisense molecules, antibodies that bind BTF4 or B7-L1 and inhibit binding to the respective receptors, compounds that block interaction of BTF4 or B7-L1 and its receptors on a immune cell (e.g., soluble, monovalent BTF4 or B7-L1 molecules, and soluble forms of BTF4 or B7-L1 receptors or compounds identified in the subject screening assays). Also included are inhibitors of the BTF4 or B7-L1 receptor (BTF4 or B7-L1 receptor antagonists), which include, without limitation: antisense molecules, antibodies that bind to BTF4 or B7-L1 receptors, but do not transduce an inhibitory signal to the immune cell (“non-activating antibodies”), and soluble forms of the BTF4 and B7-L1 receptor.

Exemplary agents for use in downmodulating an immune response by enhancing BTF4 or B7-L1 mediated signaling (BTF4 or B7-L1 agonists) include (for example): nucleic acid molecules encoding BTF4 or B7-L1 polypeptides, multivalent forms of BTF4 or B7-L1, compounds that increase the expression of BTF4 or B7-L1, and cells that express activating forms of BTF4 or B7-L1. Also included are agents for use in enhancing the activity of the BTF4 or B7-L1 receptor (BTF4 or B7-L1 receptor agonists), which include, without limitation: antibodies that bind to and activate the BTF4 or B7-L1 receptor, compounds that enhance the expression of the BTF4 or B7-L1 receptor, nucleic acid molecules encoding BTF4 or B7-L1 receptors, and activating forms of the BTF4 or B7-L1 molecule. In one embodiment, an agent engages an activating receptor (e.g., the TCR or CD3) at the same time that it engages a BTF4 or B7-L1n receptor.

C. Downregulation of Immune Responses by Enhancing BTF4 or B7-L1 Mediated Signaling

There are numerous embodiments of the invention for upregulating BTF4 or B7-L1 mediated signaling to thereby downregulate immune responses. Downregulation can be in the form of inhibiting or blocking an immune response already in progress or may involve preventing the induction of an immune response. The functions of activated immune cells can be inhibited by down-regulating immune cell responses or by inducing specific anergy in immune cells, or both. In one embodiment, the immune response is downregulated by the transmission of a signal via a receptor to which BTF4 or B7-L1 binds, to induce tolerance.

Forms of BTF4 or B7-L1 that bind to and activate the respective receptor, e.g., multivalent BTF4 or B7-L1 on a cell surface, can be used to downmodulate the immune response. Likewise, the BTF4 or B7-L1 receptor pathway can also be stimulated by the use of an agent to thereby downmodulate the immune response. One example of such an agent is an activating antibody which cross-links the BTF4 or B7-L1 receptor to provide negative signals to immune cells on which the receptors are located.

In one embodiment of the invention, an activating antibody used to stimulate receptor activity is a bispecific antibody. For example, such an antibody can comprise a receptor binding site and another binding site which targets a cell surface receptor on an immune cell, e.g., on a T cell, a B cell, or a myeloid cell. In one embodiment, such an antibody, in addition to comprising a BTF4 or B7-L1 receptor binding site can further comprise a binding site which binds to a molecule which is in proximity to the receptor, e.g., B-cell antigen receptor, a T-cell antigen receptor, or an Fc receptor in order to target the molecule to a specific cell population. For example, a CD3 antigen, a T-cell receptor chain, LFA-1, CD2, CTLA-4, immunoglobulin, B cell receptor, Ig alpha, Ig beta, CD22, or Fc receptor could be used. Such antibodies (or other bispecific agents) are art recognized and can be produced, e.g., as described herein. Selection of this second antigen for the bispecific antibody provides flexibility in selection of cell population to be targeted for inhibition.

In another embodiment, the co-ligation of the BTF4 or B7-L1 receptor and another receptor (activating or inhibitory) on a cell can enhance the generation of a negative signal via the BTF4 or B7-L1 receptor. Such co-ligation can be accomplished e.g., by use of a bispecific agent, e.g., a bispecific antibody as described herein having specificity for both receptors which are to be co-ligated. In another embodiment, the use of a multivalent form of an agent that transmits a negative signal via the BTF4 or B7-L1 receptor can be used to enhance the transmission of a negative signal via the BTF4 or B7-L1 receptor, e.g., an agent presented on a bead or on a surface. In another embodiment, such a multivalent agent can comprise two specificities to achieve co-ligation of the BTF4 or B7-L1 receptor and another receptor, or a receptor associated molecule (e.g., a bead comprising anti-CD3 and either BTF4 or B7-L1).

Agents that block or inhibit interaction of BTF4 or B7-L1 with their receptor (e.g., soluble forms of BTF4 or B7-L1 or blocking antibodies to BTF4 or B7-L1) can be identified by their ability to inhibit immune cell proliferation and/or effector function or to induce anergy when added to an in vitro assay. Agents that promote a BTF4 or B7-L1-mediated inhibitory signal or agonists of the BTF4 or B7-L1 receptor which activate the receptor (e.g., receptor activating antibodies or receptor activating small molecules) can be identified by their ability to enhance BTF4 or B7-L1 mediated signaling when added to an in vitro assay. For example, in a standard T cell activation assay, cells can be cultured in the presence of an agent that stimulates T cell activation in the presence of BTF4 or B7-L1. A number of art recognized readouts of cell activation can be employed to measure, e.g., cell proliferation or effector function (e.g., antibody production, cytokine production, phagocytosis) in the presence of the activating agent. The ability of a test agent to block or enhance the downmodulating effect of BTF4 or B7-L1 on this activation can be readily determined by measuring the ability of the agent to effect an increase, or decrease, respectively, in proliferation, or effector function being measured.

In one embodiment of the invention, tolerance is induced against specific antigens by co-administering an antigen with an agent that enhances BTF4 or B7-L1 mediated signaling. For example, tolerance can be induced to a specific antigen by increasing BTF4 or B7-L1 signaling in immune cells which are specifically activated by that antigen. This may be achieved, for example by administering the antigen in combination with an agent that increases BTF4 or B7-L1 signaling (e.g., wherein the antigen and agent are physically linked). For example, patients that receive Factor VIII frequently generate antibodies against this clotting factor. For example, co-administration of an agent that enhanced BTF4 or B7-L1 mediated signaling in combination with recombinant factor VIII (or physically linked to Factor VIII, e.g., by cross-linking) can result in downmodulation of the immune response to Factor VIII. In this way, immune responses to allergens or foreign proteins to which an immune response is undesirable can be inhibited.

In one embodiment, two separate peptides (for example, an agent which enhances BTF4 or B7-L1 mediated signaling and a second agent for inhibiting the activity of a costimulatory B lymphocyte antigen (e.g., B7-1 or B7-2), (for example, an agonist of BTF4 or B7-L1 combined with blocking antibodies to B7-2 and/or B7-1) can be combined as a single composition or administered separately (simultaneously or sequentially) to downregulate immune cell mediated immune responses in a subject. Furthermore, a therapeutically active amount of one or more agents which enhance BTF4 or B7-L1 mediated signaling can be used in conjunction with other downmodulating reagents to influence immune responses. Examples of other immunomodulating reagents include antibodies that block a costimulatory signal, (e.g., against CD28, ICOS), antibodies that activate an inhibitory signal via CTLA4, and/or antibodies against other immune cell markers (e.g., against CD40, against CD40 ligand, or against cytokines), fusion proteins (e.g., CTLA4-Fc, PD-1-Fc), and immunosuppressive drugs, (e.g., rapamycin, cyclosporine A or FK506).

The BTF4 or B7-L1 peptides, or mimics thereof, may also be useful in the construction of therapeutic agents which block immune cell function by destruction of cells. For example, portions of a BTF4 or B7-L1 polypeptide can be linked to a toxin to make a cytotoxic agent capable of triggering the destruction of cells to which it binds.

For making cytotoxic agents, polypeptides may be linked, or operatively attached, to toxins using techniques that are known in the art, e.g., crosslinking or via recombinant DNA techniques. The preparation of immunotoxins is, in general, well known in the art (see, e.g., U.S. Pat. No. 4,340,535, and EP 44167, both incorporated herein by reference). Numerous types of disulfide-bond containing linkers are known which can successfully be employed to conjugate the toxin moiety with a polypeptide. In one embodiment, linkers that contain a disulfide bond that is sterically “hindered” are to be preferred, due to their greater stability in vivo, thus preventing release of the toxin moiety prior to binding at the site of action.

A wide variety of toxins are known that may be conjugated to polypeptides or antibodies of the invention. Examples include: numerous useful plant-, fungus- or even bacteria-derived toxins, which, by way of example, include various A chain toxins, particularly ricin A chain, ribosome inactivating proteins such as saporin or gelonin, alpha.-sarcin, aspergillin, restrictocin, ribonucleases such as placental ribonuclease, angiogenic, diphtheria toxin, and pseudomonas exotoxin, etc. A preferred toxin moiety for use in connection with the invention is toxin A chain which has been treated to modify or remove carbohydrate residues, deglycosylated A chain. (U.S. Pat. No. 5,776,427).

Infusion of one or a combination of such cytotoxic agents, (e.g., BTF4 or B7-L1 ricin (alone or in combination with B7-2-ricin or B7-1-ricin), into a patient may result in the death of immune cells, particularly in light of the fact that activated immune cells that express higher amounts of the BTF4 or B7-L1 receptors. For example, because the receptor may be induced on the surface of activated lymphocytes, an antibody against receptor can be used to target the depletion of these specific cells by Fc-R dependent mechanisms or by ablation by conjugating a cytotoxic drug (e.g., ricin, saporin, or calicheamicin) to the antibody. In one embodiment, the antibody toxin can be a bispecific antibody. Such bispecific antibodies are useful for targeting a specific cell population, e.g., using a marker found only on a certain type of cell, e.g., a TCR, BCR, or FcR molecule.

Upregulating BTF4 or B7-L1 mediated signaling is useful to downmodulate the immune response, e.g., in situations of tissue, skin and organ transplantation, in graft-versus-host disease (GVHD), An individual who has a condition which involves or is precipitated by an inappropriate or abnormal immune response would benefit from the downregulation of that immune response. Such conditions include, without limitation, having an organ or tissue transplant, an allergy, or an autoimmune disorder. Examples of autoimmune diseases or disorders associated with an inappropriate or abnormal immune response include rheumatoid arthritis, juvenile rheumatoid arthritis, psoriatic arthritis, allergies, contact dermatitis, psoriasis, leprosy reversal reactions, erythema nodosum leprosum, autoimmune uveitis, multiple sclerosis, allergic encephalomyelitis, systemic lupus erythematosus, acute necrotizing hemorrhagic encephalopathy, idiopathic bilateral progressive sensorineural hearing loss, aplastic anemia, pure red cell anemia, idiopathic thrombocytopenia, polychondritis, scleroderma, Wegener's granulomatosis, chronic active hepatitis, myasthenia gravis, Stevens-Johnson syndrome, idiopathic sprue, lichen planus, Crohn's disease, Graves ophthalmopathy, sarcoidosis, primary biliary cirrhosis, primary juvenile diabetes, dry eye associated with Sjögren's syndrome, uveitis posterior, and interstitial lung fibrosis.

For example, blockage of immune cell function results in reduced tissue destruction in tissue transplantation. Typically, in tissue transplants, rejection of the transplant is initiated through its recognition as foreign by immune cells, followed by an immune reaction that destroys the transplant. The administration of a molecule which inhibits an increase in BTF4 or B7-L1 mediated signaling (such as a soluble, monomeric form of a BTF4 or B7-L1 polypeptide) alone or in conjunction with another downmodulatory agent prior to, or at the time of, transplantation can inhibit the generation of a costimulatory signal. Moreover, promotion of a BTF4 or B7-L1 signal may also be sufficient to anergize the immune cells, thereby inducing tolerance in a subject. Induction of long-term tolerance downmodulating the immune response via increased BTF4 or B7-L1 signaling may avoid the necessity of repeated administration of these reagents.

Downregulation of the immune response by an agent which increases BTF4 or B7-L1 signaling is enhanced by further contacting the immune cell with an (additional) agent which downregulates the immune response. Similarly, the antigen presenting cell may be manipulated to further cause downregulation of the immune cell response (e.g., to express additional molecules which will downregulate the immune cell response, such as PD-L1). This combination therapy may be necessary to achieve sufficient immunosuppression or tolerance in a subject, it may also be desirable to block the costimulatory function of other molecules. For example, it may be desirable to additionally block the function of other B7 family members, such as B7-1, B7-2 and/or B7-4. Blocking a B7 costimulatory activity is achieved using an agent that interferes with the ability of a B7 molecule to bind its natural ligand and/or that interferes with the ability of a B7 molecule to modulate an immune response, (e.g., a T cell response) as measured by cytokine production and/or proliferation. Exemplary agents include blocking antibodies, peptides that block the ability of the B7 molecule to bind to its natural ligand but which fail to transmit a signal through that natural ligand, peptidomimetics, small molecules, and the like. Administration may be separately or together in a single composition, and may be prior to or at the time of disease onset or transplantation.

Other downmodulatory agents that can be used in connection with the downmodulatory methods of the invention include, for example, agents that transmit an inhibitory signal via CTLA4, soluble forms of CTLA4, antibodies that activate an inhibitory signal via CTLA4, blocking antibodies against other immune cell markers or soluble forms of other receptor ligand pairs (e.g., agents that disrupt the interaction between CD40 and CD40 ligand (e.g., anti CD40 ligand antibodies), antibodies against cytokines, or immunosuppressive drugs.

Downmodulating an immune response by increasing BTF4 or B7-L1 mediated signaling is also useful in treating autoimmune disease. Many autoimmune disorders are the result of inappropriate activation of immune cells that are reactive against self tissue and which promote the production of cytokines and autoantibodies involved in the pathology of the diseases. Preventing the activation of autoreactive immune cells may reduce or eliminate disease symptoms. Administration of agents which block costimulation of immune cells by increasing BTF4 or B7-L1 mediated signaling is useful to inhibit immune cell activation and prevent production of autoantibodies or cytokines which may be involved in the disease process. Additionally, agents that increase BTF4 or B7-L1 mediated signaling may induce antigen-specific tolerance of autoreactive immune cells which could lead to long-term relief from the disease. The efficacy of reagents in preventing or alleviating autoimmune disorders can be determined using a number of well-characterized animal models of human autoimmune diseases. Examples include murine experimental autoimmune encephalitis, systemic lupus erythematosus in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Inhibition of an immune response to inhibit immune cell activation, through enhancement of BTF4 or B7-L1 mediated signaling is useful therapeutically in the treatment of allergy and allergic reactions, e.g., by inhibiting IgE production. An agent that downmodulates an immune response by increasing BTF4 or B7-L1 mediated signaling can be administered to an allergic subject to inhibit immune cell mediated allergic responses in the subject. Administration can be accompanied by exposure to allergen in conjunction with appropriate MHC molecules. Allergic reactions can be systemic or local in nature, depending on the route of entry of the allergen and the pattern of deposition of IgE on mast cells or basophils. Thus, administration may be local (e.g., to inhibit a local reaction) or systemic (e.g., to inhibit a systemic reaction).

Inhibition of an immune response to inhibit immune cell activation, through enhancement of BTF4 or B7-L1 mediated signaling may also be important therapeutically in viral infections of immune cells. For example, in the acquired immune deficiency syndrome (AIDS), viral replication is stimulated by immune cell activation. Increasing BTF4 or B7-L1 induce signaling of the infected immune cell function may result in inhibition of viral replication and thereby ameliorate the course of AIDS.

Downregulation of an immune response via enhancement of BTF4 or B7-L1 mediated signaling, may also be useful in treating an autoimmune attack of autologous tissues For example, B-7 family members (e.g., B74) are known to be normally highly expressed in the heart and protect the heart from autoimmune attack. This is evidenced by the fact that the Balb/c PD-1 knockout mouse exhibits massive autoimmune attack on the heart with thrombosis. Thus, conditions that are caused or exacerbated by autoimmune attack (e.g., in this example, heart disease, myocardial infarction or atherosclerosis) may be ameliorated or improved by increasing the activity of these B7 family members. It is therefore within the scope of the invention to modulate conditions exacerbated by autoimmune attack, such as autoimmune disorders (as well as conditions such as heart disease, myocardial infarction, and atherosclerosi) by stimulating BTF4 or B7-L1 mediated signaling.

D. Upregulation of Immune Responses

Inhibiting enhancement of BTF4 or B7-L1 mediated signaling as a means of upregulating immune responses is also useful in therapy. An individual who has a condition which involves or is precipitated by an underactive immune response would benefit from the upregulation of that immune response. Upregulation of immune responses can be in the form of enhancing an existing immune response or eliciting an initial immune response. For example, enhancing an immune response through enhancing enhancement of BTF4 or B7-L1 mediated signaling is useful in cases of infections with microbes, e.g., bacteria, viruses, or parasites, also cases of immunosuppressive disorders or the presence of a tumor. To achieve treatment of such an individual, an antagonist or other agent which inhibits BTF4 of B7-L1 mediated signaling, described herein, is administered to the patient or otherwise used in ex vivo therapy. For example, in one embodiment, administration of an agent that inhibits enhancement of BTF4 or B7-L1 mediated signaling is therapeutically useful in situations where upregulation of antibody and cell-mediated responses, resulting in more rapid or thorough clearance of virus, would be beneficial. These would include viral skin diseases such as Herpes or shingles, in which case such an agent can be delivered topically to the skin. In addition, systemic viral diseases such as influenza, the common cold, and encephalitis might be alleviated by the administration of such agents systemically.

In certain instances, it may be desirable to further administer other agents that upregulate immune responses, such as forms of other B7 family members that transduce signals via costimulatory receptors, in order to further augment the immune response. In one embodiment of the invention, upregulation of the immune response by an agent which decreases BTF4 or B7-L1 signaling, (e.g., by acting on the antigen presenting cell or the immune cell) is enhanced by further contacting the immune cell with an (additional) agent which upregulates the immune response, such as a costimulatory molecule.

Alternatively, immune responses can be enhanced in an infected patient by removing immune cells from the patient and activating the immune cells by methods which include contacting the immune cells with an agent that inhibits enhancement of BTF4 or B7-L1 mediated signaling, and reintroducing the in vitro stimulated immune cells into the patient.

Agents which inhibit BTF4 or B7-L1 mediated signaling can be used prophylactically in vaccines against various polypeptides, e.g., polypeptides derived from pathogens. Immunity against a pathogen (e.g., a virus) can be induced by vaccinating with a viral protein along with a form of the agent in an appropriate adjuvant. Alternately, a vector comprising genes which encode for both a pathogenic antigen and a form of the agent can be used for vaccination. Nucleic acid vaccines can be administered by a variety of means, for example, by injection (e.g., intramuscular, intradermal, or the biolistic injection of DNA-coated gold particles into the epidermis with a gene gun that uses a particle accelerator or a compressed gas to inject the particles into the skin (Haynes et al. 1996. J. Biotechnol. 44:37)). Alternatively, nucleic acid vaccines can be administered by non-invasive means. For example, pure or lipid-formulated DNA can be delivered to the respiratory system or targeted elsewhere, e.g., Peyers patches by oral delivery of DNA (Schubbert. 1997. Proc. Natl. Acad. Sci. USA 94:961). Attenuated microorganisms can be used for delivery to mucosal surfaces. (Sizemore et al. 1995. Science. 270:29)

In one embodiment, an agent which inhibits BTF4 or B7-L1 mediated signaling can be administered with a costimulatory molecule (e.g., a B7 family member such as B7-1, B7-2, or B74) to result in activation of T cells and provide immunity from infection. For example, pathogens for which vaccines are useful include hepatitis B, hepatitis C, Epstein-Barr virus, cytomegalovirus, HIV-1, HIV-2, tuberculosis, malaria and schistosomiasis.

In another application, upregulation of an immune response by inhibiting of BTF4 or B7-L1 mediated signaling is useful in the induction of tumor immunity. Tumor cells (e.g., sarcoma, melanoma, lymphoma, leukemia, neuroblastoma, carcinoma) transfected with a nucleic acid molecule encoding a B7 costimulatory antigen (e.g., B7-1, B7-2, or B7-4) can be administered to a subject to overcome tumor-specific tolerance in the subject. Expression of an agent that inhibits BTF4 or B7-L1 signaling (e.g., an agent that inhibits expression of BTF4 or B7-L1) by the tumor cells will further enhance tumor immunity. If desired, the tumor cell can be transfected to express a combination of B7 polypeptides (e.g., B7-1, B7-2, B7-4). For example, tumor cells obtained from a patient can be transfected ex vivo with an expression vector directing the expression of the agent and the B7 costimulatory antigen. Induction of tumor immunity may also result from expression of the agent alone, by the tumor cell. The transfected tumor cells are returned to the patient to result in expression of the encoded peptide(s) by the transfected cell. Alternatively, gene therapy techniques can be used to target a tumor cell for transfection in vivo.

In another embodiment, the immune response can be upregulated by the inhibition of signal transmission via a receptor to which BTF4 or B7-L1 binds, such that a preexisting tolerance is overcome. For example, immune responses against antigens to which a subject cannot mount a significant immune response, e.g., to an autologous antigen, such as a tumor specific antigens can be induced by administering an agent that inhibits BTF4 or B7-L1 mediated signaling. For example, in one embodiment, soluble receptor or soluble BTF4 or B7-L1 (e.g., BTF4Fc or B7-L1Fc) to block signaling and enhance an immune response, e.g., to a tumor cell. In one embodiment, an autologous antigen, such as a tumor-specific antigen can be coadministered with the agent. In another embodiment, an immune response can be stimulated against an antigen (e.g., an autologous antigen) to treat a neurological disorder. In another embodiment, the agent can be used as an adjuvant to boost responses to foreign antigens in the process of active immunization.

In yet another embodiment, the production of functional BTF4 or B7-L1 (e.g., a form of BTF4 or B7-L1 that binds the receptor) can be inhibited, e.g., using antisense RNA, in order to upregulate the immune response. For example, in one embodiment, the production of BTF4 or B7-L1 or a functional variant thereof, by a tumor cell can be inhibited in order to increase anti-tumor immunity.

In one embodiment, immune cells are obtained from a subject and cultured and activated ex vivo to in the presence of an agent that that inhibits BTF4 or B7-L1 signaling, to expand the population of activated immune cells. In a further embodiment the immune cells are then administered to a subject. Immune cells can be stimulated to proliferate in vitro by, for example, providing to the immune cells a primary activation signal and a costimulatory signal, as is known in the art. Various forms of B7 family proteins can also be used to costimulate proliferation of immune cells. In one embodiment immune cells are cultured ex vivo according to the method described in PCT Application No. WO 94/29436. The costimulatory molecule can be soluble, attached to a cell membrane or attached to a solid surface, such as a bead.

VI. Pharmaceutical Compositions

For administration to an individual, modulators of BTF4 or B7-L1 signaling (e.g., BTF4 or B7-L1 inhibitory or stimulatory agents, including BTF4 or B7-L1 nucleic acid molecules, proteins, antibodies described above, or compounds identified as modulators of a BTF4 or B7-L1 activity and/or expression or modulators of the interaction between BTF4 or B7-L1) will preferably be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a BTF4 or B7-L1 protein or anti-BTF4 or B7-L1 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Non-oral administration may alternatively be employed. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration. Kits for practice of the instant invention are also provided. For example, such a diagnostic kit comprises an antibody conjugated to a toxin. The kit can further comprise a means for administering the antibody conjugate, e.g., one or more syringes. The kit can come packaged with instructions for use.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In one embodiment of the present invention a therapeutically effective amount of an antibody which binds BTF4 or B7-L1 protein is administered to the individual. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat an individual, including but not limited to the severity of the disease or disorder, previous treatments, the general health and/or age of the subject, and other diseases present. Moreover, treatment of an individual with a therapeutically effective amount of an antibody can include a single treatment or, preferably, can include a series of treatments. In a preferred embodiment, the individual is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays as described herein.

VII. Administration of The BTF4 and B7-L1 Modulators of the Invention

Administration is preferably by means necessary to contact the appropriate cells of the individual (e.g., antigen presenting cells, immune cells such as T cells and B cells) with the agent. Which cells the agent must contact is dependent upon the particular agent used, and the particular condition to be treated, and can be determined by the skilled practitioner. The agents and modulators may be used independently or alternatively in combination to produce the desired modification of the immune response. They may also be used advantageously in combination with additional agents which likewise modulate the immune response.

The contents of all references, pending patent applications and published patents, cited throughout this application are hereby expressly incorporated by reference. Each reference disclosed herein is incorporated by reference herein in its entirety.

### EXAMPLES

### Example 1

### Generation of Fusion Proteins

Fusion proteins were generated using amino acid sequences which contained extracellular regions of either BTF4 or B7-L1 molecule. The BTF4 sequence used corresponds to the sequences reported previously as clone cc130_{—}1 (accession Number ATCC 98501) in U.S. Ser. No. 09/746,783. The B7-L1 sequence used to construct the fusion protein corresponds to the sequences reported previously as clone cr1162_{—}25 in U.S. Ser. No. 09/047,661. Note that B7-L1 is also referred to on occasion as DE01-86. Fusion proteins of both BTF4 (amino acids 1-249 of the sequence set forth in SEQ ID NO:2) and B7-L1 (amino acids 1-323 of the sequence set forth in SEQ ID NO:4) were generated by fusing the extracellular region of either molecule to the hinge-CH2-CH3 domain of human IgG1, to produce BTF4.Fc and B7-L1.Fc. The fusion proteins ICOS-L.Fc, PD-L1.Fc, B7-2.Fc were generated according to the methods of Freeman et al., (J. Exp. Med. 192: 1027-1034 (2000)) by otherwise standard methods. The resulting soluble constructs were transfected into COS cells for the production of conditioned media. This conditioned media was then purified by protein A affinity chromatography.

The BTF4.Fc fusion protein generated has the amino acid sequence shown below. The BTF4 portion is shown in bold:

(SEQ ID NO: 5)

MKMASSLAFLLLNFHVSLLLVQLLTPCSAQFSVLGPSGPILAMVGEDADL
PCHLFPTMSAETMELKWVSSSLRQVVNVYADGKEVEDRQSAPYRGRTSIL
RDGITAGKAALRIHNVTASDSGKYLCYFQDGDFYEKALVELKVAALGSNL
HVEVKGYEDGGIHLECRSTGWYPQPQIQWSNAKGENIPAVEAPVVADGVG
LYEVAASVIMLGGSGEGVSCIIRNSLLGLEKTASISIADPFFRSAQPWIG
SGSRDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPVPIEKTISKAKGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGK*

The B7-L1.Fc fusion protein generated has the amino acid sequence shown below. The B7-L1 portion is shown in bold:

(SEQ ID NO: 6)

MGAPAASLLLLLLLFACCWAPGGANLSQDDSQPWTSDETVVAGGTVVLKC
QVKDHEDSSLQWSNPAQQTLYFGEKRALRDNRIQLVTSTPHELSISISNV
ALADEGEYTCSIFTMPVRTAKSLVTVLGIPQKPIITGYKSSLREKDTATL
NCQSSGSKPAARLTWRKGDQELHGEPTRIQEDPNGKTFTVSSSVTFQVTR
EDDGASIVCSVNHESLKGADRSTSQRIEVLYTPTAMIRPDPPHPREGQKL
LLHCEGRGNPVPQQYLWEKEGSVPPLKMTQESALIFPFLNKSDSGTYGCT
ATSNMGSYKAYYTLNVNDPSPVPGSGSRDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPVPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK*

The purified fusion proteins were then assayed for their effects on T cell activation.

### Example 2

### Signaling Via BTF4 or B7-L1 Downmodulates Immune Cell Activation

In an effort to further determine the function of B7-L1 and BTF4, the effects of each protein on T cell activation were determined in a standard T cell activation assay. Human CD4⁺ T cells were purified by negative selection from peripheral blood lymphocytes. Pre-activated T cells were prepared by stimulation for 72 hours with anti-CD3/anti-CD28 beads followed by overnight rest prior to assay set up. Tosyl-activated magnetic microspheres (Dynal, Great Neck, N.Y.) were coated with anti-CD3 mAb (1 μg/10⁷ microspheres), and ICOS-L.Fc, PD-L1.Fc, B7-2.Fc, B7-L1.Fc, or BTF4.Fc (4 μg/10⁷ microspheres) according to manufacture's instructions. Murine IgG or an irrelevant fusion protein was used to saturate the binding capacity of the microspheres (total protein=5 μg/10⁷ microspheres). Anti-CD3 beads contained 1 μg/10⁷ beads anti-CD3 and 4 μg/10⁷ beads irrelevant fusion protein.

Preactivated human CD4⁺ T cells were contacted with microspheres coated with either anti-CD3 mAb and B7-L1.Fc fusion protein, anti-CD3 mAb and BTF4.Fc fusion protein, anti-CD3 mAb and ICOS-L.Fc fusion protein, anti-CD3 mAb and PD-L1.Fc fusion protein, anti-CD3 mAb and B7-2.Fc fusion protein, or anti-CD3 mAb alone. Protein-coated microspheres were added to preactivated or resting CD4⁺ T cells (10⁵ cells/well) at a ratio of 1:1 in the presence or absence of soluble anti-CD28 mAb (50 ng/ml). ICOS-L and B7-2 are known costimulatory molecules, while PF-L1 is known to inhibit T cell activation. T cell proliferation was then quantitated.

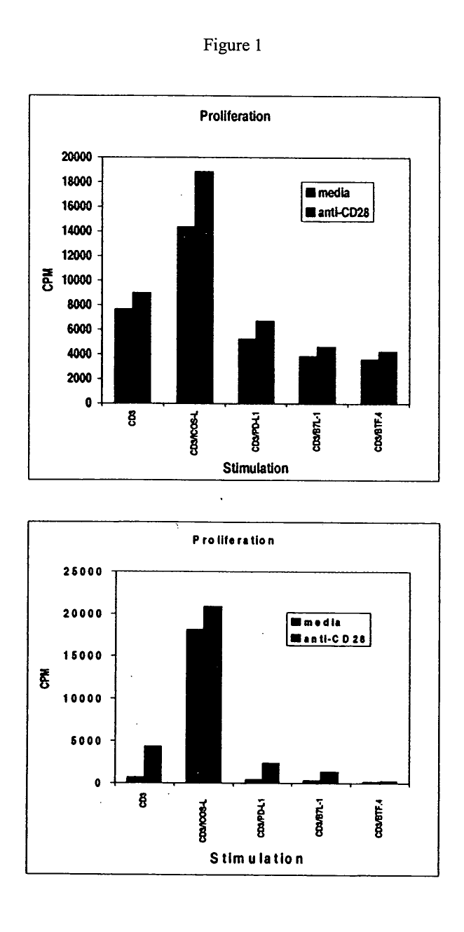

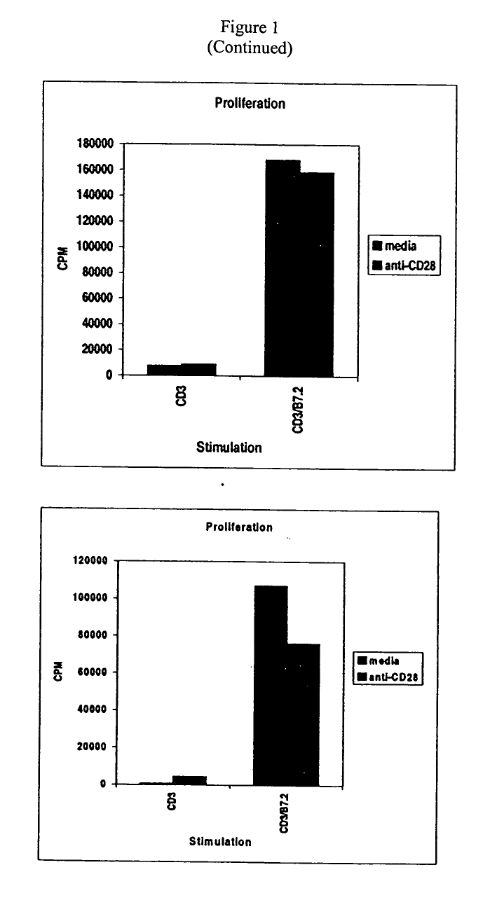

At 72 hours, proliferation was assessed by labeling cultures with 1 μCi/well tritiated thymidine and incubated for a further 6-16 hour period. Results are presented in FIG. 1. As expected stimulation of the T cells with the anti-CD3 and ICOS-L.Fc coated beads, or with the anti-CD3 and B7-2.Fc coated beads, produced substantial proliferation. This proliferation was further enhanced in the presence of soluble anti-CD28. PD-L1 is known to interact with the immunoinhibitory receptor PD-1 (Freeman, G. J. et al. (2000) J. Exp. Med. 192:1027-34). Stimulation of the T cells with the anti-CD3 and PD-L1.Fc coated beads resulted in a decrease in proliferation as compared to proliferation induced by beads coated only with anti-CD3. This decreased proliferation was only slightly enhanced by the presence of soluble anti-CD28. Stimulation of the T cells with anti-CD3 and B7-L1.Fc coated beads, or anti-CD3 and BTF4.Fc coated beads also resulted in decreased proliferation, which was only slightly enhanced by the presence of soluble anti-CD28. This is consistent with the effects of B7-L1 and BTF4 mediated signaling producing an inhibitory effect on T cell activation.

The finding that B7-L1 inhibits the proliferative response of T cells in a T cell activation assay is in contrast to previous reports (see, e.g., WO 00/08158). It is interesting to note that the inhibitory effects of B7-L1 and BTF4 were even more pronounced than the inhibitory effect of PD-L1. These results indicate that engagement of B7-L1 or BTF4 receptors expressed on T cells leads to the delivery of negative signals through these receptors to down-regulate activation through the T cell receptor. These findings indicate that development of agonists or antagonists of B7-L1 and/or BTF4 mediated signaling will lead to the development of therapeutics for modulation of T cell activation.

Equivalents

Those skilled in the art will recognize, or be able to ascertain using no more than routine experimentation, many equivalents to the specific embodiments of the invention described herein. Such equivalents are intended to be encompassed by the following claims.

1. A method for downmodulating an immune response comprising contacting an immune cell with an agent that increases BTF4 mediated signaling, to thereby downmodulate the immune response.

2. The method of claim 1, wherein the immune cell is a T cell.

3. The method of claim 1, wherein the agent is a BTF4 polypeptide.

4. The method of claim 1, wherein the agent is an extracellular portion of a BTF4 polypeptide crosslinked to an insoluble matrix.

5. The method of claim 1, wherein the agent is a BTF4-Ig fusion protein comprising the amino acid sequence shown in SEQ ID NO: 5, crosslinked to an insoluble matrix.

6. The method of claim 1, wherein the step of contacting occurs in vivo.

7. The method of claim 1, wherein the step of contacting occurs in vitro.

8. The method of claim 1, further comprising contacting the T cell with an additional agent which downregulates an immune response.

9. A method for treating a subject having a condition that would benefit from downregulation of an immune response comprising administering to the subject an agent that promotes BTF4-mediated signaling such that a condition that would benefit from downregulation of an immune response is treated.

10. The method of claim 9, wherein the subject has a condition selected from the group consisting of: a transplant, an allergy, and an autoimmune disorder.

11. The method of claim 9, wherein the agent is a BTF4 polypeptide.

12. The method of claim 9, further comprising administering a second agent which downregulates an immune response to the subject.
